(19) United States
(12) Patent Application Publication
(10) Pub. No.: US 2026/0216241 A1
(43) Pub. Date: Jul. 30, 2026

(54) CHIMERIC ANTIGEN RECEPTOR TARGETING GPC-3 AND IMMUNE CELLS EXPRESSING SUCH FOR THERAPEUTIC USES

(71) Applicant: CRISPR THERAPEUTICS AG, Zug (CH)

(72) Inventor: Jason Sagert, Cambridge, MA (US)

(21) Appl. No.: 18/872,498

(22) PCT Filed: Jun. 28, 2023
(86) PCT No.: PCT/IB2023/056715

§ 371 Date: Dec. 6, 2024

Related U.S. Application Data
(60) Provisional application No. 63/356,671, filed on Jun. 29, 2022.

(51) Int. Cl. A61K 35/17; A61K 40/11; A61K 40/31; A61K 40/42; A61P 35/00; C07K 14/705; C07K 16/30; C12N 5/0783

(52) U.S. Cl. CPC A61K 35/17; A61K 40/11; A61K 40/31; A61K 40/4202; A61P 35/00; C07K 14/7051; C07K 14/70517; C07K 14/70521; C07K 14/70578; C07K 16/303; C12N 5/0636; A61K 2239/13; A61K 2239/21; A61K 2239/22; C07K 2317/565; C07K 2317/569; C12N 2510/00

(57) ABSTRACT

Genetically engineered T cells expressing a chimeric antigen receptor (CAR) targeting GPC3 and optionally having multiple genetic edits, including a disrupted TRAC gene, a disrupted β2M gene, a disrupted Regnase I gene, a disrupted TGFbRII gene, or a combination thereof. Also provided herein are methods of making such genetically engineered T cells and methods of using the genetically engineered T cells in cancer treatment.

### CROSS-REFERENCE TO RELATED APPLICATIONS

This application is a U.S. 371 National Entry of PCT/IB2023/056715, filed Jun. 28, 2023 which claims the benefit under 35 U.S.C. § 119(e) of U.S. Provisional Patent Application No. 63/356,671, filed Jun. 29, 2022, the content of which is incorporated by reference herein in its entirety.

### SEQUENCE LISTING

The instant application contains a Sequence Listing that has been filed electronically in XML format and is hereby incorporated by reference in its entirety. Said XML copy, created on Feb. 2, 2026, is named “CRISP_42760_252_CORRECTED_SequenceListing.xml” and is 95,917 bytes in size.

### BACKGROUND OF THE INVENTION

Glypican-3 (GPC3), a 580-AA heparan sulfate proteoglycan, is overexpressed in many malignant tumors including hepatocellular carcinoma, lung squamous cell carcinoma, ovarian carcinomas, and melanoma, but is not present or shows very low expression in normal Roswell Parkssues as well as in normal liver tissue or liver cirrhosis. Capurro et al. found that 72% of HCC patients expressed GPC3, while GPC3 serum levels were significantly increased in 53% of patients with HCC. The function of GPC3 has been proposed to promote the development of HCC by activating the Wnt signaling pathway. Morgan et al. discovered that when GPC3 on HCC cells was silenced, the proliferation and invasion capacities of HCC cells were impaired, suggesting that the expression of GPC3 on HCC cells was involved in their proliferation and invasion. In addition, its overexpression is also a predictor of poor prognosis. As a consequence, due to its strong specificity and high sensitivity, GPC3 has been used as a target for the diagnosis and treatment of HCC.

Chimeric antigen receptor (CAR) T-cell therapy uses genetically modified T cells to more specifically and efficiently target and kill target cells such as target cancer cells. After T cells have been collected from the blood, the cells are engineered to include CARs on their surface. The CARs may be introduced into the T cells using CRISPR/Cas9 gene editing technology. When these CAR T cells are injected into a patient, the receptors enable the T cells to kill the target cells.

### SUMMARY OF THE INVENTION

The present disclosure is based, at least in part, on the development of genetically edited anti-GPC CAR-T cells, which may carry one or more additional genetic edits, for example, a disrupted Regnase 1 (Reg1) gene, a disrupted transforming growth factor beta receptor II (TGFbRII) gene, a disrupted Casitas B-Lineage Lymphoma Proto-Oncogene-B (chl-b) gene, a disrupted T cell receptor alpha chain constant region (TRAC) gene, and/or a disrupted beta-2 microglobulin (β2M) gene, and effective methods of producing such genetically edited T cells via CRISPR/Cas-mediated gene editing using guide RNAs as disclosed herein.

Accordingly, in some aspects, the present disclosure features a population of genetically engineered T cells, wherein the genetically engineered T cells comprise: (a) a nucleic acid encoding a chimeric antigen receptor (CAR) that binds Glypican 3 (GPC3) (anti-GPC3 CAR); and (b) one or more disrupted genes, which comprise: (i) a disrupted T cell receptor alpha chain constant region (TRAC) gene, (ii) a disrupted beta-2 microglobulin (β2M) gene, (iii) a disrupted transforming growth factor beta receptor II (TGFbRII) gene, (iv) a disrupted Regnase-1 (Reg1) gene, (v) a disrupted Casitas B-Lineage Lymphoma Proto-Oncogene-B (CBLB) gene, or (vi) a combination of any one of (i)-(v). In some instances, the T cells are human T cells. In some examples, the T cells comprise primary human T cells.

In some embodiments, the anti-GPC3 CAR comprises: (a) an ectodomain that binds GPC3; (b) a transmembrane domain; and (c) an endodomain that comprises (i) a co-stimulatory signaling domain and (ii) a CD3ζ cytoplasmic signaling domain.

In some instances, the ectodomain comprises an anti-GPC3 fragment, which is an anti-GPC3 single domain antibody (e.g., VHH). Such an anti-GPC3 single domain antibody may comprise the same complementarity determining regions (CDRs) as those in SEQ ID NO: 9. In one example, the anti-GPC3 VHH comprises the amino acid sequence of SEQ ID NO: 9.

In some instances, the ectodomain comprises an anti-GPC3 fragment, which is an anti-GPC3 single chain variable fragment (scFv). In some examples, the ectodomain comprises the anti-GPC3 scFv, which comprises a heavy chain variable (V_{H}) region comprising the same CDRs as those in SEQ ID NO: 22, and a light chain variable (V_{L}) region comprising the same CDRs as those in SEQ ID NO: 21. In specific examples, the anti-GPC3 scFv comprises the V_{H} set forth as SEQ ID NO: 22, and a V_{L} set forth as SEQ ID NO: 21. Such an anti-GPC3 scFv may comprise the amino acid sequence of SEQ ID NO: 23. Alternatively, the anti-GPC3 scFv may comprise the amino acid sequence of SEQ ID NO: 24.

Any of the anti-GPC3 CARs disclosed herein may comprise a co-stimulatory domain. In some examples, the co-stimulatory domain is a CD28 co-stimulatory domain. In other examples, the co-stimulatory domain is a 4-1BB co-stimulatory domain. Alternatively or in addition, the anti-GPC3 CAR may comprise a transmembrane domain, which can be a CD8 transmembrane domain. In specific examples, the anti-GPC3 CAR comprises an amino acid sequence of SEQ ID NOs: 11, 14, 26, 29, 32, or 35. In one example, the anti-GPC3 CAR comprises the amino acid sequence of SEQ ID NO: 11. In another example, the anti-GPC3 CAR comprises the amino acid sequence of SEQ ID NO: 26.

In some embodiments, the genetically engineered T cells may comprise the disrupted TRAC gene and the disrupted, β2M gene. Such genetically engineered T cells may further comprise the disrupted TGFBRII gene, the disrupted Reg-1 gene, the disrupted CBLB gene, or a combination thereof. For example, the genetically engineered T cells may further comprise (i) the disrupted TGFBRII gene and the disrupted Reg-1 gene. Alternatively, the genetically engineered T cells may further comprise the disrupted TGFBRII gene and the disrupted CBLB gene.

In other embodiments, the genetically engineered T cells may comprise the disrupted TGFBRII gene, the disrupted Reg-1 gene, the disrupted CBLB gene, or a combination thereof. In some examples, such genetically engineered T cells may comprise a wild-type TRAC gene, a wild-type β2M gene, or a combination thereof.

In some instances, the nucleic acid encoding the anti-GPC3 CAR may be inserted in an endogenous genetic locus of the genetically engineered T cells. For example, the endogenous genetic locus is within the disrupted TRAC gene, the disrupted, β2M gene, the disrupted TGFbRII gene, the disrupted Reg-1 gene, or the disrupted CBLB gene. In some examples, the endogenous genetic locus is within the disrupted TRAC gene. In specific examples, the disrupted TRAC gene comprises a deletion of SEQ ID NO: 58, which may be replaced by the nucleotide sequence encoding the anti-GPC3 CAR.

In other aspects, provided herein is a method for producing a population of genetically engineered T cells, the method comprising: (a) delivering to a population of T cells (i) one or more RNA-guided nucleases, (ii) one or more guide RNAs targeting a T cell receptor alpha chain constant region (TRAC) gene (TRAC gRNA), a beta-2 microglobulin (β2M) gene (β2M gRNA), a TGFbRII gene (TGFBRII gRNA), a Regnase-1 (Reg1) gene (Reg1 gRNA), and/or a Casitas B-Lineage Lymphoma Proto-Oncogene-B (CBLB) gene (CBLB gRNA); and (iii) a vector comprising a nucleic acid encoding an anti-GPC3 CAR such as those disclosed herein; and (b) producing a population of engineered T cell expressing the anti-GPC3 CAR and comprises one or more of disrupted TRAC gene, β2M gene, TGFBRII gene, Reg1 gene, and CBLB gene.

In some examples, the population of T cells comprise human T cells, e.g., human primary T cells. In some examples, the population of T cells is obtained from one or more healthy human donors. Alternatively, the population of T cells is obtained from a human patient having a GPC3+ cancer.

In some embodiments, step (a) comprises delivering to the population of T cells the TRAC gRNA and the β2M gRNA. In some instances, step (a) further comprises delivering to the population of T cells the TGFBRII gRNA, the Reg1 guide, the CBLB guide, or a combination thereof. For example, step (a) may further comprise delivering to the population of T cells (i) the TGFBRII gRNA and the Reg1 guide. Alternatively, step (a) may comprise delivering to the population of T cells the TGFBRII guide and the CBLB guide. In other instances, step (a) comprises (e.g., consists of) delivering to the population of T cells the TGFBRII gRNA, the Reg1 guide, the CBLB guide, or a combination thereof.

In some examples, the TRAC guide is specific to a TRAC gene target sequence comprising the nucleotide sequence of SEQ ID NO:58. Such a TRAC guide may comprise a spacer comprising the nucleotide sequence of SEQ ID NO: 39. In some instances, the TRAC guide may comprise a scaffold sequence (e.g., those disclosed herein). In some instances, the TRAC guide may comprise one or more modifications. Examplary TRAC guides may comprise the nucleotide sequence of SEQ ID NO: 37, or SEQ ID NO: 38.

In some examples, the β2M guide is specific to a β2M gene target sequence comprising the nucleotide sequence of SEQ ID NO: 60. Such a β2M guide may comprise a spacer comprising the nucleotide sequence of SEQ ID NO: 43. In some instances, the β2M guide may comprise a scaffold sequence (e.g., those disclosed herein). In some instances, the β2M guide may comprise one or more modifications. Exemplary β2M guide may comprise the nucleotide sequence of SEQ ID NO: 41, or SEQ ID NO: 42.

In some examples, the TGFBRII guide is specific to a TGFBRII gene target sequence comprising the nucleotide sequence of SEQ ID NO: 62. Such a TGFBRII guide may comprise a spacer comprising the nucleotide sequence of SEQ ID NO: 47. In some instances, the TGFBRII guide may comprise a scaffold sequence (e.g., those disclosed herein). In some instances, the TGFBRII guide may comprise one or more modifications. Exemplary TGFBRII guide may comprise the nucleotide sequence of SEQ ID NO: 45, or SEQ ID NO: 46.

In some examples, the Reg1 guide is specific to a Reg1 gene target sequence comprising the nucleotide sequence of SEQ ID NO: 64. Such a Reg1 guide may comprises a spacer comprising the nucleotide sequence of SEQ ID NO: 51. In some instances, the Reg1 guide may comprise a scaffold sequence (e.g., those disclosed herein). In some instances, the Reg1 guide may comprise one or more modifications. Exemplary Reg1 guide may comprise the nucleotide sequence of SEQ ID NO: 49, or SEQ ID NO: 50.

In some examples, the CBLB guide is specific to a CBLB gene target sequence comprising the nucleotide sequence of SEQ ID NO: 66. Such a CBLB guide may comprise a spacer comprising the nucleotide sequence of SEQ ID NO: 55. In some instances, the CBLB guide may comprise a scaffold sequence (e.g., those disclosed herein). In some instances, the CBLB guide may comprise one or more modifications. Exemplary CBLB guide may comprise the nucleotide sequence of SEQ ID NO: 53, or SEQ ID NO: 54.

In any of the methods disclosed herein, the one or more RNA-guided nucleases may comprise a Cas9 nuclease. In some examples, the Cas9 nuclease is a S. pyogenes Cas9 nuclease.

Alternatively or in addition, the vector of (a)(iii) for use in any of the methods disclosed herein may comprise a donor template in which the nucleic acid encoding the anti GPC3 CAR is flanked by an upstream fragment and a downstream fragment, and wherein the upstream fragment and the downstream fragment are homologous to an endogenous genetic locus of the T cells, allowing for insertion of the nucleic acid encoding the anti-GPC3 CAR into the endogenous genetic locus. Exemplary endogenous genetic loci include, but are not limited to, the disrupted TRAC gene, the disrupted β2M gene, the disrupted TGFbRII gene, the disrupted Reg-1 gene, or the disrupted CBLB gene. In some examples, the endogenous genetic locus is within the disrupted TRAC gene. In that case, the upstream fragment may be SEQ ID NO: 71, and/or the downstream fragment may be SEQ ID NO: 74.

In some examples, the vector of (a)(iii) is a viral vector, for example, an adeno associated viral (AAV) vector or a lentiviral vector.

In other aspects, provided herein is a method of treating cancer in a subject, comprising administering to a subject in need thereof any of the populations of genetically engineered T cells disclosed herein. In some embodiments, the subject is a human patient having a GPC3+ cancer. Exemplary target cancers include, but are not limited to, a liver cancer, a gastric cancer, a colorectal cancer, a lung cancer, an ovarian cancer, a skin cancer, or a thyroid cancer. In one example, the target cancer is HCC.

In some examples, the population of genetically engineered T cells is allogeneic to the human patient. Such a population of genetically engineered T cells may have a disrupted TRAC gene and/or a disrupted β2M gene, and optionally a disrupted TGFBRII gene, a disrupted Reg-1 gene, and/or a disrupted cbl-b gene. Alternatively, the population of genetically engineered T cells is autologous to the human patient. Such a population of genetically engineered T cells may have a disrupted TGFBRII gene, a disrupted Reg-1 gene, and/or a disrupted cbl-b gene, and optionally wild-type TRAC and/or β2M genes.

Further, the present disclosure provides a chimeric antigen receptor that binds Glypican 3 (GPC3) (anti-GPC3 CAR) as disclosed herein. In some instances, the anti-GPC3 CAR may further comprise an N-terminus signal peptide. Exemplary anti-GPC3 CAR polypeptides (with or without the signal peptide) are provided in Table 1, all of which are within the scope of the present disclosure. Also provided herein are nucleic acids encoding the anti-GPC3 CAR and host cells comprising such nucleic acids. In some instances, the nucleic acids are located in a suitable vector, for example, a viral vector such as an AAV vector or a lentiviral vector.

In addition, provided herein are any of the anti-GPC3 CAR-expressing genetically engineered T cells for use in inhibiting GPC3+ disease cells such as cancer cells and for treating a disease associated with such disease cells, for example, the various cancers disclosed herein. Also within the scope of the present disclosure are the anti-GPC3 CAR expressing genetically engineered T cells in manufacturing a medicament for use in the intended therapeutic applications.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1B include diagrams showing assessment of anti-GPC3 CAR expression and editing efficiency TRAC and B2M (β2 Microglobulin) genes. FIG. 1A: a graph showing expression of anti-GPC3 CAR in T cells transduced with the indicated CAR constructs. FIG. 1B: a graph showing efficiency of TRAC and B2M gene KO in T cells expressing the indicated CAR constructs.

FIGS. 2A-2B include diagrams showing frequency of CD4+ and CD8+ T cells, and assessment of relative enrichment of T cell populations from CCR7/CD45RA staining. FIG. 2A: a graph showing frequency of CD4+ and CD8+ T cells in the cell cohorts expressing the indicated CAR constructs. FIG. 2B: a graph showing relative enrichment of Teff, Tcm, Tnaive and TEMRA in the cell cohorts.

FIG. 3 is a graph showing expression of immune checkpoint molecules Programmed cell death protein 1 (PD1) and Lymphocyte-activation gene 3 (Lag3), in T cells expressing the indicated CAR constructs.

FIGS. 4A-4C include diagrams showing cytotoxic effects of the anti-GPC3 CAR expressing T cells. FIG. 4A: a graph showing cytotoxicity of anti-GPC3 CAR T cells on GPC3^{Hi} HepG2 target cells at the indicated T-cell/Target cell ratios. FIG. 4B: a graph showing cytotoxicity of anti-GPC3 CAR T cells on GPC3^{Lo} Huh7 target cells at the indicated T-cell/Target cell ratios. FIG. 4C: a graph showing cytotoxicity of anti-GPC3 CAR T cells on GPC3⁻ A498 target cells at the indicated T-cell/Target cell ratios.

FIG. 5 is a graph showing the effects of anti-GPC3 CAR T cell administration on tumor volumes in NSG mice implanted with GPC3⁺ HepG2 cancer cells.

FIG. 6 is a graph showing s in vitro expansion of anti-GPC3 CAR T cells post electroporation.

FIG. 7 is a graph showing assessment of anti-GPC3 CAR expression in TRAC⁻/B2M⁻/anti-GPC3 CAR T cells with additional disruptions of TGFbRII/Reg-1 (T+R) or TGFbRII/cbl-b (T+C)

FIGS. 8A-8C include diagrams showing show editing efficiency for TRAC, B2M, TGFbRII and Regnase-1 (Reg-1) genes in the CAR T cells described in FIG. 7. FIG. 8A: a graph showing efficiency of TRAC and B2M gene KO in the CAR T cells. FIG. 8B: a graph showing efficiency of TGFbRII gene KO in the TGFbRII/Reg-1 (T+R) CAR T cells. FIG. 8C: a graph showing efficiency of Reg-1 gene KO in the TGFbRII/Reg-1 (T+R) CAR T cells.

FIG. 9 is a graph showing efficiency of cbl-b KO by western blot analysis in the TGFbRII/cbl-b KO (T+C) CAR T cells described in FIG. 7.

FIG. 10 is a graph showing the frequency of CD4+ and CD8+ T cells in the CAR T cells described in FIG. 7.

FIGS. 11A-11B include diagrams showing cytotoxic effects of the CAR T cells described in FIG. 7. FIG. 11A: a graph showing cytotoxicity of the anti-GPC3 CAR T cells on GPC3^{Hi} HepG2 target cells at the indicated T-cell/Target cell ratios. FIG. 11B: a graph showing cytotoxicity of anti-GPC3 CAR T cells on GPC3⁻ A498 target cells at the indicated T-cell/Target cell ratios.

FIGS. 12A-12B include diagrams showing assessment of cytokine secretion by the CAR T cells that were incubated with the GPC3^{Hi} HepG2 target cells, described in FIG. 11A. FIG. 12A: a graph showing IFN-gamma secretion. FIG. 12B: a graph showing Granzyme A secretion.

### DETAILED DESCRIPTION OF THE INVENTION

The present disclosure aims at establishing genetically engineered anti-GPC3 CAR-T cells having improved features, such as growth activity, persistence, reduced T cell exhaustion, and/or enhanced potency. The anti-GPC3 CAR-T cells disclosed herein may comprise multiple genetic edits on endogenous genes, for example, disruption of the TRAC gene, the β2M gene, the TGFBRII gene, the Reg1 gene, and/or the cbl-b gene to make the cells suitable for either allogeneic immune cell therapy or autologous immune cell therapy and to achieve features that could improve treatment efficacy. For example, β2M disruption can reduce the risk of or prevent a host-versus-graft response and TRAC disruption can reduce the risk of or prevent a graft-versus-host response. Accordingly, the anti-GPC3 CAR-T cells disclosed herein, having disrupted TRAC gene and/or disrupted β2M gene may be suitable for use in allogeneic cell therapy. Alternatively or in addition, TGFBRII disruption may reduce immunosuppressive effect of transforming growth factor beta (TGF-β) in the tumor microenvironment and Reg1 disruption may improve CAR-T cell functionality via long-term persistence with robust effector function. Further, CAR-T cells with a disrupted cblb gene as disclosed herein also showed enhanced anti-tumor activity and prolonged survival rates. In some instances, the anti-GPC3 CAR-T cells having disrupted TGFBRII gene, Reg1 gene, and/or cbl-b gene with wild-type TRAC and β2M genes may be suitable for use in autologous cell therapy.

As provided herein, the anti-GPC3 CAR-T cells disclosed herein showed high editing efficiencies of target genes such as TRAC, β2M, Reg-1, TGFBRII, and/or cbl-b and no significant interference with expression of immune checkpoint molecules such as PD1 and LAG-3. Further, the anti-GPC3 CAR-T cells exhibited high cytotoxicity against GPC3+ cells and GPC3+ tumor both in vitro and in vivo. The additional gene edits, e.g., disruption of Reg-1, TGFBRII, and/or cbl-b genes showed no impact on CAR-T cell properties, including cell growth and CD4:CD8 cell ratios. CAR-T cells bearing the additional gene edits showed good proliferation and specific cytotoxicy to targets and were deemed phenotypically normal. Further, such CAR-T cells exhibited enhanced anti-tumor activities as observed in animal models. As such, the anti-GPC3 CAR-T cells disclosed herein, such as those having one or more of the gene edits (disruption of Reg-1, TGFBRII, and/or cbl-b genes), would be expected to achieve superior anti-tumor efficacy, either in autologous cell therapy or allogenic cell therapy.

Such a T cell may use bonafide T cells as the starting material, for example, non transformed T cells, terminally differentiated T cells, T cells having stable genome, and/or T cells that depend on cytokines and growth factors for proliferation and expansion. Alternatively, such a T cell may use T cells generated from precursor cells such as hematopoietic stem cells (e.g., iPSCs), e.g., in vitro culture. The T cells disclosed herein may confer one or more benefits in both CAR-T cell manufacturing and clinical applications.

Other advantageous features associated with the disruption of Reg1 gene and/or the TGFBRII gene may be found in WO/2022/064428, the relevant disclosures of which are incorporated by reference for the subject matter and purpose referenced herein.

Accordingly, provided herein are anti-GPC3 CAR-T cells, which may have improved persistence and enhanced anti-tumor activity, methods of producing such T cells, and therapeutic applications of such T cells in eliminating GPC3+ disease cells such as cancer cells (e.g., GPC3+ hepatocellular carcinoma cells).

### I. Anti-GPC3 CAR-T Celis Having Enhanced Features

Glypican-3 (GPC3) is known to be an important cell surface marker for many malignant tumors including hepatocellular carcinoma, lung squamous cell carcinoma, ovarian carcinomas, and melanoma. Due to its strong specificity and high sensitivity, GPC3 has been used as a target for the diagnosis and treatment of such GPC3+ tumors such as liver cancer, gastric cancer, colorectal cancer, lung cancer, ovarian cancer, skin cancer, and a thyroid cancer.

In some aspects, provided herein are anti-GPC3 CAR-T cells, which may have multiple genetic modifications to improve CAR-T cell functionality and thus therapeutic efficacy for either allogenic cell therapy or autologous cell therapy. The genetically engineered T cells provided herein express a chimeric antigen receptor (CAR) that binds GPC3 and optionally have multiple genetic edits on endogenous genes, for example, on a TRAC gene, a β2M gene, a Reg1 gene, a TGFBRII gene, or a combination thereof. In some instances, the anti-GPC3 CAR-T cells disclosed herein comprise disrupted TRAC and, β2M genes, as well as Reg1, TGFBRII, and/or cbl-b genes. In other instances, the anti-GPC3 CAR-T cells disclosed herein comprise disrupted Reg1, TGFBRII, and/or cbl-b genes and may have wild-type TRAC and β2M genes.

The genetically engineered T cells may be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors. Alternatively, the parent T cells may be differentiated from precursor T cells obtained from one or more suitable donor or stem cells such as hematopoietic stem cells or inducible pluripotent stem cells (iPSC), which may be cultured in vitro.

Any of the genetically engineered T cells may be generated via gene editing (including genomic editing), a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

### A. Genetically Edited Genes

In some aspects, the present disclosure provides genetically engineered T cells that may comprise a disrupted Reg1 gene, a disrupted TGFBRII gene, a disrupted cbl-b gene, a disrupted TRAC gene, and/or a disrupted β2M gene.

As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

### Reg1 Gene Editing

In some embodiments, the genetically engineered T cells may comprise a disrupted gene involved in mRNA decay. Such a gene may be Reg1. Reg1 contains a zinc finger motif, binds RNA and exhibits ribonuclease activity. Reg1 plays roles in both immune and non immune cells and its expression can be rapidly induced under diverse conditions including microbial infections, treatment with inflammatory cytokines and chemical or mechanical stimulation. Human Reg1 gene is located on chromosome 1p34.3. Additional information can be found in GenBank under Gene ID: 80149.

In some examples, the genetically engineered T cells may comprise a disrupted Reg1 gene such that the expression of Reg1 in the T cells is substantially reduced or eliminated completely. The disrupted Reg1 gene may comprise one or more genetic edits at one or more suitable target sites (e.g., in coding regions or in non-coding regulatory regions such as promoter regions) that disrupt expression of the Reg1 gene. Such target sites may be identified based on the gene editing approach for use in making the genetically engineered T cells. Exemplary target sites for the genetic edits may include exon 1, exon 2, exon 3, exon 4, exon 5, exon 6, or a combination thereof. In some examples, one or more genetic editing may occur in exon 2 or exon 4. Such genetic editing may be induced by the CRISPR/Cas technology using a suitable guide RNA, for example, those listed in Table 2. See also WO/2022/064428, the relevant disclosures of which are incorporated by reference for the subject matter and purpose referenced herein.

Disruption of the Reg1 gene can enhance long-term-persistence and maintain robust effector function, thereby improving T cell functionality.

### TGFBRII Gene Editing

In some embodiments, the genetically engineered T cells may comprise a disrupted TGFBRII gene, which encodes Transforming Growth Factor Receptor Type II (TGFBRII). TGFBRII receptors are a family of serine/threonine kinase receptors involved in the TGFβ signaling pathway. These receptors bind growth factor and cytokine signaling proteins in the TGFβ family, for example, TGFβs (TGFβ1, TGFβ2, and TGFβ3), bone morphogenetic proteins (BMPs), growth differentiation factors (GDFs), activin and inhibin, myostatin, anti-Müllerian hormone (AMH), and NODAL.

In some examples, the genetically engineered T cells may comprise a disrupted TGFBRII gene such that the expression of TGFBRII in the T cells is substantially reduced or eliminated completely. The disrupted TGFBRII gene may comprise one or more genetic edits at one or more suitable target sites (e.g., in coding regions or in non-coding regulatory regions such as promoter regions) that disrupt expression of the TGFBRII gene. Such target sites may be identified based on the gene editing approach for use in making the genetically engineered T cells. Exemplary target sites for the genetic edits may include exon 1, exon 2, exon 3, exon 4, exon 5, or a combination thereof. In some examples, one or more genetic editing may occur in exon 4 and/or exon 5. Such genetic editing may be induced by a gene editing technology, (e.g., the CRISPR/Cas technology) using a suitable guide RNA, for example, those listed in Table 2. See also WO/2022/064428, the relevant disclosures of which are incorporated by reference for the subject matter and purpose referenced herein.

Disruption of the TGFBRII gene can eliminate surface expression of TGFBRII and reduce the immunosuppressive effect of transforming growth factor beta (TFG-β) in the tumor microenvironment.

cbl-b Gene Editing

In some embodiments, the genetically engineered T cells may comprise a disrupted Cbl proto-oncogene B (cbl-b) gene. The CBLB protein contains a zinc finger motif, binds RNA and exhibits ribonuclease activity. CBLB plays roles in both immune and non-immune cells and its expression can be rapidly induced under diverse conditions including microbial infections, treatment with inflammatory cytokines and chemical or mechanical stimulation. Human cbl-b gene is located on chromosome GRCh38.p13. Additional information can be found in GenBank under Gene ID: 868.

In some examples, the genetically engineered T cells may comprise a disrupted cbl-b gene such that the expression of cbl-b in the T cells is substantially reduced or eliminated completely. The disrupted cbl-b gene may comprise one or more genetic edits at one or more suitable target sites (e.g., in coding regions or in non-coding regulatory regions such as promoter regions) that disrupt expression of the cbl-b gene. Such target sites may be identified based on the gene editing approach for use in making the genetically engineered T cells. Exemplary target sites for the genetic edits may include exon 2, exon 7, exon 9, exon 11, exon 12, or a combination thereof. In some examples, one or more genetic editing may occur in exon 2. In other examples, one or more genetic editing may occur in exon 7. In yet other examples, one or more genetic editing may occur in exon 9. Such genetic editing may be induced by the CRISPR/Cas technology using a suitable guide RNA, for example, those listed in Table 2. See also U.S. Provisional Application No. 63/292,715, the relevant disclosures of which are incorporated by reference for the subject matter and purpose referenced herein.

### β2M Gene Edit

In some embodiments, the genetically engineered T cells disclosed herein may further comprise a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus graft response.

In some embodiments, an edited β2M gene may comprise a nucleotide sequence selected from the following sequences in Table 2. It is known to those skilled in the art that different nucleotide sequences in an edited gene such as an edited β2M gene may be generated by a single gRNA such as the one listed in Table 2 (2M-1). See also WO2019097305, the relevant disclosures of which are incorporated by reference for the subject matter and purpose referenced herein.

### TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein.

Such genetic editing of the TRAC gene may be induced by a gene editing technology, (e.g., the CRISPR/Cas technology) using a suitable guide RNA, for example, those listed in Table 2.

It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.

In some instances, a nucleic acid encoding an anti-GPC3 CAR may be inserted into the TRAC gene, thereby disrupting expression of the TRAC gene. For example, the CAR-coding nucleic acid may replace the target site of a gRNA used in gene editing via CRISPR/Cas9 (e.g., replacing the fragment comprising SEQ ID NO: 58 in the TRAC gene.

### B. Anti-GPC3 Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. An exemplary signal peptide is provided in Table 1. Other signal peptides may be used.

### (i) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some instances, the extracellular antigen binding domain may be an antibody fragment that binds GPC3 (e.g., human GPC3), for example, a single chain variable fragment (scFv) or a single domain antibody fragment such as a heavy chain-only antibody fragment (VHH).

Anti-GPC3 scFv Fragments

In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

In some embodiments, the antigen-binding extracellular domain can be a single-chain variable fragment (scFv) that binds the GPC3 antigen as disclosed herein. The scFv may comprise an antibody heavy chain variable region (V_{H}) and an antibody light chain variable region (V_{L}), which optionally may be connected via a flexible peptide linker. In some instances, the scFv may have the V_{H} to V_{L} orientation (from N-terminus to C-terminus). Alternatively the scFv may have the V_{L} to V_{H} orientation (from N-terminus to C-terminus).

In some examples, the antigen-binding extracellular domain can be a single-chain variable fragment (scFv) that binds human GPC3. In some instances, the anti-GPC3 scFv may comprises (i) a heavy chain variable region (V_{H}) that comprises the same heavy chain complementary determining regions (CDRs) as those in SEQ ID NO:22; and (ii) a light chain variable region (V_{L}) that comprises the same light chain CDRs as those in SEQ ID NO: 21. See Table 1 below. In some specific examples, the anti-GPC3 antibody disclosed herein may comprise the heavy chain CDR1, heavy chain CDR2, and heavy chain CDR3 set forth as SEQ ID NOs: 18-20, respectively as determined by the Kabat method. Alternatively or in addition, the anti-GPC3 antibody discloses herein may comprise the light chain CDR1, light chain CDR2, and light chain CDR3 set forth as SEQ ID NOs: 15-17 as determined by the Kabat method. In one specific example, the anti-GPC3 scFv may comprise a V_{H} comprising the amino acid sequence of SEQ ID NO: 22 and a V_{L} comprises the amino acid sequence of SEQ ID NO: 21. See Table 1 below.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.bioinf.org.uk/abs/.

In other embodiments, an anti-GPC3 scFv may be a functional variant derived from the exemplary anti-GPC3 antibody listed in Table 1. Such a functional variant is substantially similar to the exemplary anti-GPC3 antibody both structurally and functionally. A functional variant comprises substantially the same V_{H} and V_{L} CDRs as Ab-1 or Ab-2. For example, it may comprise only up to 8 (e.g., 8, 7, 6, 5, 4, 3, 2, or 1) amino acid residue variations in the total CDR regions relative to the exemplary anti-GPC3 antibody and binds the same epitope of GPC3 with substantially similar affinity (e.g., having a K_{D} value in the same order). In some instances, the functional variants may have the same heavy chain CDR3 as the exemplary anti-GPC3 antibody, and optionally the same light chain CDR3 as the exemplary anti-GPC3 antibody. Such an anti-GPC3 scFv may comprise a V_{H} fragment having CDR amino acid residue variations (e.g., up to 5, for example, 5, 4, 3, 2, and 1) in only the heavy chain CDR1 and/or CDR2 as compared with the exemplary anti-GPC3 antibody. Alternatively or in addition, the anti-scFv antibody may further comprise a V_{L} fragment having CDR amino acid residue variations (e.g., up to 5, for example, 5, 4, 3, 2, and 1) in only the light chain CDR1 and/or CDR2 as compared with the exemplary anti-GPC3 antibody. In some examples, the amino acid residue variations can be conservative amino acid residue substitutions.

In some examples, any of the variations in one or more of the CDR regions can be conservative substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-GPC3 scFv derived from the exemplary anti-GPC3 antibody may be in the format of, from N-terminus to C-terminus, V_{H}-linker-V_{L}. In some examples, the anti-GPC3 scFv comprises a V_{H} fragment of SEQ ID NO: 22 and a V_{L} fragment of SEQ ID NO: 21. In specific examples, the anti-GPC3 scFv in any of the anti-GPC3 CAR may comprise the amino acid sequence of SEQ ID NO: 23. Alternatively, the anti-GPC3 scFv derived from the exemplary anti-GPC3 antibody may be in the format of, from N-terminus to C-terminus, V_{L}-linker-V_{H}. In some examples, the anti-GCP3 scFv in any of the anti-GPC3 CAR may comprise the amino acid sequence of SEQ ID NO: 24. In some instances, the anti-GPC3 scFv may share at least 85% sequence identity (e.g., at least 90%, at least 95% or above) to SEQ ID NO: 23 or SEQ ID NO: 24.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

### Anti-GPC3 Single Domain Antibody Fragments

In some embodiments, the antigen binding domain can be a single-domain antibody fragment. Single-domain antibodies, also known as nanobodies, are small antigen-binding fragments containing only one heavy or light chain variable region (as opposed to conventional antibodies having both heavy and light chain variable regions). In some instances, the single domain antibodies provided herein are heavy chain only antibodies (VHH antibodies) containing a single heavy chain variable region.

Like conventional antibodies, a single domain antibody such as a VHH antibody contain regions of hypervariability, also known as “complementarity determining regions” (“CDR”), interspersed with regions that are more conserved, which are known as “framework regions” (“FR”). A VHH antibody is typically composed of three CDRs and four FRs, arranged from amino-terminus to carboxy-terminus in the following order. FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, the anti-GPC3 antibody moiety disclosed herein may share the same complementary determining regions (CDRs) as the exemplary anti-GPC3 VHH antibody provided in Table 1 herein. Such an anti-GPC3 VHH antibody may have CDR1, CDR2, and CDR3 comprising SEQ ID NOs: 6-8, respectively (following the Kabat definition). In some embodiments, the anti-GPC3 antibody moiety disclosed herein may share a certain level of sequence identity (e.g., at least 80% such as at least 85%, at least 90%, at least 95% or above) as compared with the exemplary anti-GPC3 VHH antibody. In some embodiments, the anti-GPC3 antibody moiety disclosed herein may have one or more amino acid variations (e.g., up to 10, 9, 8, 7, 6, 5, 4, 3, 2, or 1 amino acid variations) relative to the exemplary anti-GPC3 VHH antibody. The amino acid residue variations as disclosed in the present disclosure (e.g., in framework regions and/or in CDRs) can be conservative amino acid residue substitutions. In one example, the anti-GPC3 antibody moiety may comprise the amino acid sequence of SEQ ID NO: 9.

### (ii) Transmembrane Domain

The anti-GPC3 CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the anti-GPC3 CAR containing such.

In some embodiments, the transmembrane domain of an anti-GPC3 CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a extracellular+transmembrane domain containing the sequence of SEQ ID NO: 2 as provided below in Table 1. Other transmembrane domains may be used.

(iii) Hinge Domain

In some embodiments, a hinge domain may be located between an extracellular domain (comprising the antigen binding domain) and a transmembrane domain of an anti-GPC3 CAR, or between a cytoplasmic domain and a transmembrane domain of the anti-GPC3 CAR. A hinge domain can be any oligopeptide or polypeptide that functions to link the transmembrane domain to the extracellular domain and/or the cytoplasmic domain in the polypeptide chain. A hinge domain may function to provide flexibility to the anti-GPC3 CAR, or domains thereof, or to prevent steric hindrance of the anti-GPC3 CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of an anti-GPC3 CAR. In some embodiments, the hinge domain may be a CD8 hinge domain. Other hinge domains may be used.

### (iv) Intracellular Signaling Domains

Any of the anti-GPC3 CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3 is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the anti-GPC3 CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 1 provides examples of signaling domains derived from 4-1BB, CD28 and CD3-zeta that may be used herein.

### (v) Exemplary Anti-GPC3 CAR Polypeptides

Exemplary anti-GPC3 CAR polypeptides are provided in Table 1 below, all of which are within the scope of the present disclosure (including both mature anti-GPC3 CARs, i.e., without N-terminus signal peptide, and presurcor anti-GPC3 CARs, i.e., with the N-terminus signal peptide. Also within the scope of the present disclosure are nucleic acids coding for any of the anti-GPC3 CAR constructs disclosed herein, e.g., those disclosed in Table 1. The nucleic acids may be located in a suitable vector, for example, a viral vector such as an AAV vector or a lentiviral vector. Host cells comprising such a nucleic acid, or a vector are also within the scope of the present disclosure.

### C. Methods of Making Genetically Engineered T Cells

The genetically engineered T cells disclosed herein can be prepared by genetic editing of parent T cells or precursor cells thereof via a conventional gene editing method or those described herein.

### (a) T Cells

In some embodiments, T cells can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRαβ, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some instances, the T cell population comprises primary T cells isolated from one or more human donors. Such T cells are terminally differentiated, not transformed, depend on cytokines and/or growth factors for growth, and/or have stable genomes.

Alternatively, the T cells may be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

### (b) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95: e52118).

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration. Some exemplary approaches are disclosed in detail below.

### CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

### Endonuclease for Use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). One example is provided in Table 2 below (SEQ ID NO: 70).

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence ranges from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO: 58), then the gRNA spacer sequence is 5′-AGAGCAACAGUGCUGUGGCC**-3′ (SEQ ID NO: 39). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20 nucleotides spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 2 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a Reg1 gene, for example, target a site within exon 1, exon 2, exon 3, exon 4, exon 5, or exon 6 of the Reg1 gene. Such a gRNA may comprise a spacer sequence complementary (complete or partially) to the target sequences in exon 2 or exon 4 of a Reg1 gene, or a fragment thereof. Exemplary target sequences of Reg1 and exemplary gRNA sequences are provided in Table 2 below.

In some embodiments, the gRNAs disclosed herein target a TGFBRII gene, for example, target a site within exon 1, exon 2, exon 3, exon 4, exon 5, or exon 6 of the TGFBRII gene. Such a gRNA may comprise a spacer sequence complementary (complete or partially) to the target sequences in exon 4 or exon 5 of a TGFBRII gene, or a fragment thereof. Exemplary target sequences of TGFBRII and exemplary gRNA sequences are provided in Table 2 below.

In some embodiments, the gRNAs disclosed herein target a cbl-b gene, for example, target a site within exon 2, exon 7, exon 9, exon 11, or exon 12 of the cbl-b gene. Such a gRNA may comprise a spacer sequence complementary (complete or partially) to the target sequences in exon 2 of a cbl-b gene, or a fragment thereof. In other examples, a gRNA may comprise a spacer sequence complementary (complete or partially) to the target sequences in exon 7 of a cbl-b gene, or a fragment thereof. Alternatively, a gRNA may comprise a spacer sequence complementary (complete or partially) to the target sequences in exon 9 of a cbl-b gene, or a fragment thereof. Exemplary target sequences in a cbl-b gene and exemplary gRNA sequences are provided in Table 2 below.

In some embodiments, the gRNAs disclosed herein target a A2M gene, for example, target a suitable site within a A2M gene. See also WO2019097305, the relevant disclosures of which are incorporated by reference herein for the purpose and subject matter referenced herein. Other gRNA sequences may be designed using the A2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein. Exemplary spacer sequences and gRNAs targeting a β2M gene are provided in Table 2 below.

In some embodiments, the gRNAs disclosed herein target a TRAC gene. See also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein. Exemplary spacer sequences and gRNAs targeting a TRAC gene are provided in Table 2 below.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187(1990).

In some embodiments of the present disclosure, a CRISPR/Cas nuclease system for use in genetically editing any of the target genes disclosed here may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

### Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and an RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used. In some instances, a Cas9 enzyme may form one RNP with all of the gRNAs targeting the TRAC gene, the β2M gene, the Reg1 gene, and the TGFBRII gene and be delivered to T cells via one electroporation event. Alternatively, a Cas9 enzyme may form two or more RNPs, which collectively include all of the gRNAs targeting the TRAC gene, the β2M gene, the Reg1 gene, and the TGFBRII gene. The multiple RNPs may be delivered to the T cells via sequential electroporation events, for example, two sequential electroporation events.

In other embodiments, viral vectors such as one or more lentiviral vector can be used to deliver a nucleic acid encoding the nuclease and optionally one or more gRNAs to a target cell (e.g., a T cell) for genetic editing of one or more of the target genes disclosed herein.

### Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the nucleases disclosed herein, including a CRISPR/Cas nuclease, may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

### D. Delivery of Anti-GPC3 CAR Construct to T Cells

In some embodiments, a nucleic acid encoding an anti-GPC3 CAR can be introduced into any of the genetically engineered T cells disclosed herein by methods known to those of skill in the art. For example, a coding sequence of the anti-GPC3 CAR may be cloned into a vector, which may be introduced into the genetically engineered T cells for expression of the anti-GPC3 CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In some examples, a nucleic acid encoding an anti-GPC3 CAR construct can be delivered to a cell using an adeno-associated virus (AAV). AAVs are small viruses which integrate site-specifically into the host genome and can therefore deliver a transgene, such as the anti-GPC3 CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the anti-GPC3 CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding an anti-GPC3 CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions disclosed herein can be used for this purpose.

In some examples, a genomic deletion in the TRAC gene and replacement by an anti-GPC3 CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting an anti-GPC3 CAR coding segment into the TRAC gene.

A donor template as disclosed herein can contain a coding sequence for an anti-GPC3 CAR. In some examples, the anti-GPC3 CAR-coding sequence may be flanked by two regions of homology to allow for efficient HDR at a genomic location of interest, for example, at a TRAC gene using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous prompter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO: 72 provided in Table 3 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, a donor template for delivering an anti-GPC3 CAR may be an AAV vector inserted with a nucleic acid fragment comprising the coding sequence of the anti-GPC3 CAR, and optionally regulatory sequences for expression of the anti-GPC3 CAR (e.g., a promoter such as the EF1a promoter provided in Table 3), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 58. In some specific examples, the donor template for delivering the anti-GPC3 CAR may comprise a nucleotide sequence set forth in SEQ ID NOs: 12, 75, 27, 30, 33, or 36, which may franked by the upstream and downstream homology arms (e.g., SEQ ID NO: 71 and SEQ ID NO: 74). In some instances, the nucleic acid encoding the CAR can be inserted into a disrupted TRAC gene, for example, replacing the fragment of SEQ ID NO: 58.

In other examples, a nucleic acid encoding an anti-GPC3 CAR construct can be delivered to a cell using a lentiviral vector. Lenvirival vectors can infect both dividing and non-dividing cells. As such, they can deliver transgenes to non-proliferating or slowly proliferating cells efficiently, making them attractive for clinical applications. Lentiviral vectors carrying a nucleic acid encoding any of the anti-GPC3 CARs disclosed herein can be constructed following conventional methods. See also Example 5 below.

E. Exemplary Anti-GPC3 CAR-T Cells Optionally with Multiple Genetic Edits

In some embodiments, a population of genetically engineered T cells disclosed herein express an anti-GPC3 CAR as those disclosed herein (e.g., those provided in Table 1). Such genetically engineered T cells may also comprise a disrupted TRAC gene, a disrupted β2M gene, a disrupted Reg1 gene, a disrupted TGFBRII gene, a disrupted cbl-b gene, or a combination thereof). The nucleotide sequence encoding the anti-GPC3 CAR may be inserted in a genetic site of interest, for example, in the disrupted TRAC gene (e.g., replacing the site targeted by a sgRNA listed in Table 2 below).

In some examples, a population of genetically engineered T cells disclosed herein express an anti-GPC3 CAR as those disclosed herein (e.g., those provided in Table 1) and comprise a disrupted TRAC gene and a disrupted β2M gene. The nucleotide sequence encoding the anti-GPC3 CAR may be inserted in a genetic site of interest, for example, the disrupted TRAC gene (e.g., replacing the site targeted by a sgRNA listed in Table 2 below). Such a population of genetically engineered T cells may further comprise a disrupted Reg1 gene, a disrupted TGFBRII gene, a disrupted cbl-b gene, or a combination thereof. For example, the population of genetically engineered T cells may further comprise a disrupted Reg1 gene and a disrupted TGFBRII gene. Alternatively, the population of genetically engineered T cells may further comprise a disrupted TGFBRII gene and a disrupted cbl-b gene. In other examples, the population of genetically engineered T cells may further comprise a disrupted cbl-b gene. Such genetically engineered T cells may further comprise additional gene edits, for example, disrupted Reg-1 and/or TGFBRII genes. Alternatively, the genetically engineered T cells may have wild-type Reg-1 and/or TGFBRII genes.

In some embodiments, a population of genetically engineered T cells disclosed herein expresses an anti-GPC3 CAR as those disclosed herein (e.g., those provided in Table 1) and comprises a disrupted Reg1 gene, a disrupted TGFBRII gene, a disrupted cbl-b gene, or a combination thereof). In some examples, the population of genetically engineered T cells express the anti-GPC3 CAR and comprises a disrupted Reg1 gene and a disrupted TGFBRII gene. Alternatively, the population of genetically engineered T cells may comprise a disrupted TGFBRII gene and a disrupted cbl-b gene. In other examples, the population of genetically engineered T cells may further comprise a disrupted cbl-b gene. Such genetically engineered T cells may further comprise additional gene edits, for example, disrupted Reg-1 and/or TGFBRII genes. Alternatively, the genetically engineered T cells may have wild-type Reg-1 and/or TGFBRII genes. In some instances, the genetically engineered T cells may have a wild-type TRAC gene, a wild-type β2M gene, or both.

In some examples, the population of genetically engineered T cells may comprise about 50%-99% (e.g., about 55% to about 80%) CAR⁺ T cells, and optionally about 90%-99.9% (e.g., about 95% to about 99.7%) TCR⁻ T cells, about 50% to about 90% (e.g., about 60% to about 80%) β2M⁻ T cells, about 50% to about 90% (e.g., about 60% to about 70%) of TGFBRII⁻ T cells, about 50% to about 90% (e.g., about 60% to about 70%) Reg1⁻ T cells, and/or about 50% to about 90% (e.g., about 60% to about 70%) CBLB⁻ T cells In other examples, the population of genetically engineered T cells may comprise about 50% to about 90% (e.g., about 60% to about 70%) of TGFBRII⁺ T cells, and about 50% to about 90% (e.g., about 60% to about 70%) Reg1⁻ T cells. In yet other examples, the population of genetically engineered T cells may comprise about 50% to about 90% (e.g., about 60% to about 70%) of TGFBRII⁺ T cells, and about 50% to about 90% (e.g., about 60% to about 70%) CBLB⁺ T cells.

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein. On the other hand, a cell is deemed positive (+) in expressing a surface receptor (e.g., an anti-GPC3 CAR) when the surface expression of such a receptor can be detected via a routine method, e.g., by flow cytometry or immune staining.

Any of the anti-GPC3 CAR-T cells disclosed herein may be suspended in a cryopreservation solution (e.g., CryoStor® C55) to form a pharmaceutical composition. The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

### H. CAR-T Cell Therapy of GPC3+ Cancer

The anti-GPC3 CAR-T cells disclosed herein may be used for eliminating disease cells that express GPC3 such as GPC3+ cancer cells. For example, an effective amount of the anti-GPC3 CAR-T cells may be administered to a subject in need of the treatment via a suitable route, e.g., intravenous infusion.

The step of administering may include the placement (e.g., transplantation) of the anti-GPC3 CAR-T cells into a subject by a method or route that results in at least partial localization of the CAR-T cells at a desired site, such as a tumor site, such that a desired effect(s) can be produced. The anti-GPC3 CAR-T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life-time of the subject, i.e., long-term engraftment. For example, in some aspects described herein, an effective amount of the therapeutic T cells can be administered via a systemic route of administration, such as an intraperitoneal or intravenous route.

In some embodiments, the anti-GPC3 CAR-T cells are administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

A subject may be any subject for whom diagnosis, treatment, or therapy is desired. In some embodiments, the subject is a mammal. In some embodiments, the subject is a human. In some instances, the anti-GPC3 CAR-T cells may be autologous (“self”) to the subject, i.e., the cells are from the same subject. Alternatively, the anti-GPC3 CAR-T cells can be non-autologous (“non-self,” e.g., allogeneic, syngeneic, or xenogeneic) to the subject. “Allogeneic” means that the anti-GPC3 CAR-T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used. In some embodiments, the anti-GPC3 CAR-T cell population being administered according to the methods described herein comprises allogeneic T cells obtained from one or more donors, for example, one or more healthy human donors.

An effective amount refers to the amount of the anti-GPC3 CAR-T cells disclosed herein needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment using the anti-GPC3 CAR-T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the anti-GPC3 CAR-T cells as disclosed herein can be used to eliminate GPC3* cancer cells and/or treating a GPC3* cancer in a human patient. In some instances, the human patient may a liver cancer (e.g., HCC), a gastric cancer, a colorectal cancer, a lung cancer, an ovarian cancer, a skin cancer, or a thyroid cancer.

Combination therapies are also encompassed by the present disclosure. For example, the therapeutic T cells disclosed herein may be co-used with other therapeutic agents, for treating the same indication, or for enhancing efficacy of the therapeutic T cells and/or reducing side effects of the therapeutic T cells.

### IV. KS

The present disclosure also provides kits for use in producing the genetically engineered T cells, the therapeutic T cells, and for therapeutic uses,

In some embodiments, a kit provided herein may comprise components for performing genetic edit of one or more of the TRAC gene, the β2M gene, the TGFBRII gene, the Reg-1 gene and the cbl-b gene, and optionally a population of immune cells to which the genetic editing will be performed (e.g., a leukopak). A leukopak sample may be an enriched leukapheresis product collected from peripheral blood. It typically contains a variety of blood cells including monocytes, lymphocytes, platelets, plasma, and red cells. The components for genetically editing one or more of the target genes may comprise a suitable endonuclease such as an RNA-guided endonuclease and one or more nucleic acid guides, which direct cleavage of one or more suitable genomic sites by the endonuclease. For example, the kit may comprise a Cas enzyme such as Cas 9 and one or more gRNAs targeting a cbl-b gene. Any of the gRNAs specific to these target genes can be included in the kit. Such a kit may further comprise components for further gene editing, for example, gRNAs and optionally additional endonucleases for editing other target genes such as Reg-1, TGFBRII, cbl-b, β2M and/or TRAC.

In some embodiments, a kit provided herein may comprise a population of genetically engineered T cells as disclosed herein, and one or more components for producing the therapeutic T cells as also disclosed herein. Such components may comprise an endonuclease suitable for gene editing and a nucleic acid coding for a CAR construct of interest. The CAR-coding nucleic acid may be part of a donor template as disclosed herein, which may contain homologous arms flanking the CAR-coding sequence. In some instances, the donor template may be carried by a viral vector such as an AAV vector or a lentiviral vector.

The kit may further comprise gRNAs specific to a TRAC gene for inserting the CAR-coding sequence into the TRAC gene. In other examples, the kit may further comprise gRNAs specific to a β2M gene for inserting the CAR-coding sequence into the β2M gene. In other examples, the kit may further comprise gRNAs specific to a TGFBRII gene for inserting the CAR-coding sequence into the TGFBRII gene. In other examples, the kit may further comprise gRNAs specific to a Reg-1 gene for inserting the CAR-coding sequence into the Reg-1 gene. In yet other examples, the kit may further comprise gRNAs specific to a cbl-b gene for inserting the CAR-coding sequence into the cbl-b gene.

In yet other embodiments, the kit disclosed herein may comprise a population of therapeutic T cells as disclosed for the intended therapeutic purposes.

Any of the kit disclosed herein may further comprise instructions for making the therapeutic T cells, or therapeutic applications of the therapeutic T cells. In some examples, the included instructions may comprise a description of using the gene editing components to genetically engineer one or more of the target genes disclosed herein. In other examples, the included instructions may comprise a description of how to introduce a nucleic acid encoding a CAR construction into the T cells for making therapeutic T cells.

Alternatively, the kit may further comprise instructions for administration of the therapeutic T cells as disclosed herein to achieve the intended activity, e.g., eliminating disease cells targeted by the CAR expressed on the therapeutic T cells. The kit may further comprise a description of selecting a subject suitable for treatment based on identifying whether the subject is in need of the treatment. The instructions relating to the use of the therapeutic T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the therapeutic T cells are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the therapeutic T cells. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### SEQUENCE TABLES

TABLE 1
Anti-GPC3 CAR Components and Sequences Thereof

| | | | SEQ |
|---|---|---|---|
| Description | | Sequence | ID NO |
| signal peptide | | MALPVTALLLPLALLLHAARP | 1 |
| CD8a (extracellular + | | FVPVFLPAKPTTTPAPRPPTPAPTIASQPLSLRPEACR | 2 |
| transmembrane | | PAAGGAVHTRGLDFACDIYIWAPLAGTCGVLLLSL | |
| domain) | | VITLYCNHRNR | |
| CD28 Costimulatory | | SKRSRLLHSDYMNMTPRRPGPTRKHYQPYAPPRDF | 3 |
| Domain | | AAYRS | |
| 4-1BB Costimulatory | | KRGRKKLLYIFKQPFMRPVQTTQEEDGCSCRFPEEE | 4 |
| Domain | | EGGCEL | |
| CD3-zeta Signaling | | RVKFSRSADAPAYQQGQNQLYNELNLGRREEYDV | 5 |
| Domain | | LDKRRGRDPEMGGKPRRKNPQEGLYNELQKDKMA | |
| | | EAYSEIGMKGERRRGKGHDGLYQGLSTATKDTYD | |
| | | ALHMQALPPR | |
| Anti-GPC3 | HCDR1 | SYEM | 6 |
| VHH | HCDR2 | SIYHSGSTYYNPSLKS | 7 |
| 1524 | HCDR3 | VNMDRFDY | 8 |
| | VHH | QVQLVQSGGGLVQPGGSLRLSCAASYFDFDSYEMS | 9 |
| | | WVRQAPGKGLEWIGSIYHSGSTYYNPSLKSRVTISR | |
| | | DNSKNTLYLQMNTLRAEDTATYYCARVNMDRFDY | |
| | | WGQGTLVTVSS | |
| Anti-GPC3 CAR 1524 | | MALPVTALLLPLALLLHAARPQVQLVQSGGGLVQPG | 10 |
| (VHH; | | GSLRLSCAASYFDFDSYEMSWVRQAPGKGLEWIGS | |
| CD28 CoS | | IYHSGSTYYNPSLKSRVTISRDNSKNTLYLQMNTLR | |
| with signal peptide) | | AEDTATYYCARVNMDRFDYWGQGTLVTVSSSAAA | |
| | | FVPVFLPAKPTTTPAPRPPTPAPTIASQPLSLRPEACR | |
| | | PAAGGAVHTRGLDFACDIYIWAPLAGTCGVLLLSL | |
| | | VITLYCNHRNRSKRSRLLHSDYMNMTPRRPGPTRK | |
| | | HYQPYAPPRDFAAYRSRVKFSRSADAPAYQQGQNQ | |
| | | LYNELNLGRREEYDVLDKRRGRDPEMGGKPRRKN | |
| | | PQEGLYNELQKDKMAEAYSEIGMKGERRRGKGHD | |
| | | GLYQGLSTATKDTYDALHMQALPPR | |
| Anti-GPC3 CAR 1524 | | QVQLVQSGGGLVQPGGSLRLSCAASYFDFDSYEMS | 11 |
| (no signal peptide) | | WVRQAPGKGLEWIGSIYHSGSTYYNPSLKSRVTISR | |
| | | DNSKNTLYLQMNTLRAEDTATYYCARVNMDRFDY | |
| | | WGQGTLVTVSSSAAAFVPVFLPAKPTTTPAPRPPTP | |
| | | APTIASQPLSLRPEACRPAAGGAVHTRGLDFACDIYI | |
| | | WAPLAGTCGVLLLSLVITLYCNHRNRSKRSRLLHSD | |
| | | YMNMTPRRPGPTRKHYQPYAPPRDFAAYRSRVKFS | |
| | | RSADAPAYQQGQNQLYNELNLGRREEYDVLDKRR | |
| | | GRDPEMGGKPRRKNPQEGLYNELQKDKMAEAYSE | |
| | | IGMKGERRRGKGHDGLYQGLSTATKDTYDALHMQ | |
| | | ALPPR | |
| Anti-GPC3 CAR 1524 | | ATGGCGCTTCCGGTGACAGCACTGCTCCTCCCCTT | 12 |
| coding sequence | | GGCGCTGTTGCTCCACGCAGCAAGGCCGCAAGTG | |
| | | CAGCTCGTGCAAAGCGGCGGCGGACTGGTGCAAC | |
| | | CCGGCGGATCTCTGAGACTGAGCTGCGCCGCCAG | |
| | | CTACTTCGACTTCGACAGCTACGAGATGAGCTGG | |
| | | GTGAGGCAAGCCCCCGGAAAAGGACTGGAGTGG | |
| | | ATCGGCAGCATCTACCACAGCGGCTCCACCTACT | |
| | | ACAACCCCTCTCTGAAATCTAGAGTGACCATCTC | |
| | | TAGAGACAACTCCAAGAACACACTGTACCTCCAG | |
| | | ATGAACACACTGAGAGCCGAGGACACCGCCACCT | |
| | | ATTACTGCGCTAGAGTGAACATGGACAGATTCGA | |
| | | CTACTGGGGCCAAGGCACACTGGTGACAGTGAGC | |
| | | TCCAGTGCTGCTGCCTTTGTCCCGGTATTTCTCCC | |
| | | AGCCAAACCGACCACGACTCCCGCCCCGCGCCCT | |
| | | CCGACACCCGCTCCCACCATCGCCTCTCAACCTCT | |
| | | TAGTCTTCGCCCCGAGGCATGCCGACCCGCCGCC | |
| | | GGGGGTGCTGTTCATACGAGGGGCTTGGACTTCG | |
| | | CTTGTGATATTTACATTTGGGCTCCGTTGGCGGGT | |
| | | ACGTGCGGCGTCCTTTTGTTGTCACTCGTTATTAC | |
| | | TTTGTATTGTAATCACAGGAATCGCTCAAAGCGG | |
| | | AGTAGGTTGTTGCATTCCGATTACATGAATATGA | |
| | | CTCCTCGCCGGCCTGGGCCGACAAGAAAACATTA | |
| | | CCAACCCTATGCCCCCCCACGAGACTTCGCTGCG | |
| | | TACAGGTCCCGAGTGAAGTTTTCCCGAAGCGCAG | |
| | | ACGCTCCGGCATATCAGCAAGGACAGAATCAGCT | |
| | | GTATAACGAACTGAATTTGGGACGCCGCGAGGAG | |
| | | TATGACGTGCTTGATAAACGCCGGGGGAGAGACC | |
| | | CGGAAATGGGGGGTAAACCCCGAAGAAAGAATC | |
| | | CCCAAGAAGGACTCTACAATGAACTCCAGAAGG | |
| | | ATAAGATGGCGGAGGCCTACTCAGAAATAGGTAT | |
| | | GAAGGGCGAACGACGACGGGGAAAAGGTCACGA | |
| | | TGGCCTCTACCAAGGGTTGAGTACGGCAACCAAA | |
| | | GATACGTACGATGCACTGCATATGCAGGCCCTGC | |
| | | CTCCCAGA | |
| Anti-GPC3 CAR 1524b | | MALPVTALLLPLALLLHAARPQVQLVQSGGGLVQP | 13 |
| (VHH; | | GGSLRLSCAASYFDFDSYEMSWVRQAPGKGLEWIG | |
| 4-1BB CoS | | SIYHSGSTYYNPSLKSRVTISRDNSKNTLYLQMNTL | |
| with signal peptide) | | RAEDTATYYCARVNMDRFDYWGQGTLVTVSSSAA | |
| | | AFVPVFLPAKPTTTPAPRPPTPAPTIASQPLSLRPEAC | |
| | | RPAAGGAVHTRGLDFACDIYIWAPLAGTCGVLLLS | |
| | | LVITLYCNHRNRKRGRKKLLYIFKQPFMRPVQTTQE | |
| | | EDGCSCRFPEEEEGGCELRVKFSRSADAPAYQQGQ | |
| | | NQLYNELNLGRREEYDVLDKRRGRDPEMGGKPRR | |
| | | KNPQEGLYNELQKDKMAEAYSEIGMKGERRRGKG | |
| | | HDGLYQGLSTATKDTYDALHMQALPPR | |
| Anti-GPC3 CAR 1524b | | QVQLVQSGGGLVQPGGSLRLSCAASYFDFDSYEMS | 14 |
| (no signal peptide) | | WVRQAPGKGLEWIGSIYHSGSTYYNPSLKSRVTISR | |
| | | DNSKNTLYLQMNTLRAEDTATYYCARVNMDRFDY | |
| | | WGQGTLVTVSSSAAAFVPVFLPAKPTTTPAPRPPTP | |
| | | APTIASQPLSLRPEACRPAAGGAVHTRGLDFACDIYI | |
| | | WAPLAGTCGVLLLSLVITLYCNHRNRKRGRKKLLY | |
| | | IFKQPFMRPVQTTQEEDGCSCRFPEEEEGGCELRVK | |
| | | FSRSADAPAYQQGQNQLYNELNLGRREEYDVLDK | |
| | | RRGRDPEMGGKPRRKNPQEGLYNELQKDKMAEAY | |
| | | SEIGMKGERRRGKGHDGLYQGLSTATKDTYDALH | |
| | | MQALPPR | |
| Anti-GPC3 CAR 1524b | | ATGGCGCTTCCGGTGACAGCACTGCTCCTCCCCTT | 75 |
| coding sequence | | GGCGCTGTTGCTCCACGCAGCAAGGCCGCAAGTG | |
| | | CAGCTCGTGCAAAGCGGCGGCGGACTGGTGCAAC | |
| | | CCGGCGGATCTCTGAGACTGAGCTGCGCCGCCAG | |
| | | CTACTTCGACTTCGACAGCTACGAGATGAGCTGG | |
| | | GTGAGGCAAGCCCCCGGAAAAGGACTGGAGTGG | |
| | | ATCGGCAGCATCTACCACAGCGGCTCCACCTACT | |
| | | ACAACCCCTCTCTGAAATCTAGAGTGACCATCTC | |
| | | TAGAGACAACTCCAAGAACACACTGTACCTCCAG | |
| | | ATGAACACACTGAGAGCCGAGGACACCGCCACCT | |
| | | ATTACTGCGCTAGAGTGAACATGGACAGATTCGA | |
| | | CTACTGGGGCCAAGGCACACTGGTGACAGTGAGC | |
| | | TCCAGTGCTGCTGCCTTTGTCCCGGTATTTCTCCC | |
| | | AGCCAAACCGACCACGACTCCCGCCCCGCGCCCT | |
| | | CCGACACCCGCTCCCACCATCGCCTCTCAACCTCT | |
| | | TAGTCTTCGCCCCGAGGCATGCCGACCCGCCGCC | |
| | | GGGGGTGCTGTTCATACGAGGGGCTTGGACTTCG | |
| | | CTTGTGATATTTACATTTGGGCTCCGTTGGCGGGT | |
| | | ACGTGCGGCGTCCTTTTGTTGTCACTCGTTATTAC | |
| | | TTTGTATTGTAATCACAGGAATCGCAAACGGGGC | |
| | | AGAAAGAAACTCCTGTATATATTCAAACAACCAT | |
| | | TTATGAGACCAGTACAAACTACTCAAGAGGAAGA | |
| | | TGGCTGTAGCTGCCGATTTCCAGAAGAAGAAGAA | |
| | | GGAGGATGTGAACTGCGAGTGAAGTTTTCCCGAA | |
| | | GCGCAGACGCTCCGGCATATCAGCAAGGACAGA | |
| | | ATCAGCTGTATAACGAACTGAATTTGGGACGCCG | |
| | | CGAGGAGTATGACGTGCTTGATAAACGCCGGGGG | |
| | | AGAGACCCGGAAATGGGGGGTAAACCCCGAAGA | |
| | | AAGAATCCCCAAGAAGGACTCTACAATGAACTCC | |
| | | AGAAGGATAAGATGGCGGAGGCCTACTCAGAAA | |
| | | TAGGTATGAAGGGCGAACGACGACGGGGAAAAG | |
| | | GTCACGATGGCCTCTACCAAGGGTTGAGTACGGC | |
| | | AACCAAAGATACGTACGATGCACTGCATATGCAG | |
| | | GCCCTGCCTCCCAGA | |
| Anti- | LCDR1 | KSSQSLLYSSNQKNYLA | 15 |
| GPC3 | LCDR2 | WASSRES | 16 |
| scFv | LCDR3 | QQYYNYPLT | 17 |
| | HCDR1 | KNAMN | 18 |
| | HCDR2 | RIRNKTNNYATYYADSVKA | 19 |
| | HCDR3 | GNSFAY | 20 |
| | VL | DIVMTQSPDSLAVSLGERATINCKSSQSLLYSSNQK | 21 |
| | | NYLAWYQQKPGQPPKLLIYWASSRESGVPDRFSGS | |
| | | GSGTDFTLTISSLQAEDVAVYYCQQYYNYPLTFGQ | |
| | | GTKLEIK | |
| | VH | EVQLVESGGGLVQPGGSLRLSCAASGFTFNKNAMN | 22 |
| | | WVRQAPGKGLEWVGRIRNKTNNYATYYADSVKA | |
| | | RFTISRDDSKNSLYLQMNSLKTEDTAVYYCVAGNS | |
| | | FAYWGQGTLVTVSA | |
| | scFv | EVQLVESGGGLVQPGGSLRLSCAASGFTFNKNAMN | 23 |
| | (VH/VL) | WVRQAPGKGLEWVGRIRNKTNNYATYYADSVKA | |
| | Linker | RFTISRDDSKNSLYLQMNSLKTEDTAVYYCVAGNS | |
| | underlined | FAYWGQGTLVTVSAGGGGSGGGGSGGGGSDIVMT | |
| | | QSPDSLAVSLGERATINCKSSQSLLYSSNQKNYLAW | |
| | | YQQKPGQPPKLLIYWASSRESGVPDRFSGSGSGTDF | |
| | | TLTISSLQAEDVAVYYCQQYYNYPLTFGQGTKLEIK | |
| | scFv | DIVMTQSPDSLAVSLGERATINCKSSQSLLYSSNQK | 24 |
| | (VL/VH) | NYLAWYQQKPGQPPKLLIYWASSRESGVPDRESGS | |
| | Linker | GSGTDFTLTISSLQAEDVAVYYCQQYYNYPLTFGQ | |
| | underlined | GTKLEIKGGGGSGGGGSGGGGSEVQLVESGGGLVQ | |
| | | PGGSLRLSCAASGFTFNKNAMNWVRQAPGKGLEW | |
| | | VGRIRNKTNNYATYYADSVKARFTISRDDSKNSLY | |
| | | LQMNSLKTEDTAVYYCVAGNSFAYWGQGTLVTVS | |
| | | A | |

| | | |
|---|---|---|
| Anti-GPC3 CAR 1525 | MALPVTALLLPLALLLHAARPEVQLVESGGGLVQPGG | 25 |
| (VH/VL; CD28 CoS peptide) | SLRLSCAASGFTFNKNAMNWVRQAPGKGLEWVGR | |
| with signal | IRNKTNNYATYYADSVKARFTISRDDSKNSLYLQM | |
| | NSLKTEDTAVYYCVAGNSFAYWGQGTLVTVSAGG | |
| | GGSGGGGSGGGGSDIVMTQSPDSLAVSLGERATINC | |
| | KSSQSLLYSSNQKNYLAWYQQKPGQPPKLLIYWAS | |
| | SRESGVPDRFSGSGSGTDFTLTISSLQAEDVAVYYC | |
| | QQYYNYPLTFGQGTKLEIKSAAAFVPVFLPAKPTTT | |
| | PAPRPPTPAPTIASQPLSLRPEACRPAAGGAVHTRGL | |
| | DFACDIYIWAPLAGTCGVLLLSLVITLYCNHRNRSK | |
| | RSRLLHSDYMNMTPRRPGPTRKHYQPYAPPRDFAA | |
| | YRSRVKFSRSADAPAYQQGQNQLYNELNLGRREEY | |
| | DVLDKRRGRDPEMGGKPRRKNPQEGLYNELQKDK | |
| | MAEAYSEIGMKGERRRGKGHDGLYQGLSTATKDT | |
| | YDALHMQALPPR | |
| Anti-GPC3 CAR 1525 | EVQLVESGGGLVQPGGSLRLSCAASGFTFNKNAMN | 26 |
| (no signal peptide) | WVRQAPGKGLEWVGRIRNKTNNYATYYADSVKA | |
| | RFTISRDDSKNSLYLQMNSLKTEDTAVYYCVAGNS | |
| | FAYWGQGTLVTVSAGGGGSGGGGSGGGGSDIVMT | |
| | QSPDSLAVSLGERATINCKSSQSLLYSSNQKNYLAW | |
| | YQQKPGQPPKLLIYWASSRESGVPDRFSGSGSGTDF | |
| | TLTISSLQAEDVAVYYCQQYYNYPLTFGQGTKLEIK | |
| | SAAAFVPVFLPAKPTTTPAPRPPTPAPTIASQPLSLRP | |
| | EACRPAAGGAVHTRGLDFACDIYIWAPLAGTCGVL | |
| | LLSLVITLYCNHRNRSKRSRLLHSDYMNMTPRRPGP | |
| | TRKHYQPYAPPRDFAAYRSRVKFSRSADAPAYQQG | |
| | QNQLYNELNLGRREEYDVLDKRRGRDPEMGGKPR | |
| | RKNPQEGLYNELQKDKMAEAYSEIGMKGERRRGK | |
| | GHDGLYQGLSTATKDTYDALHMQALPPR | |
| Anti-GPC3 CAR 1525 | ATGGCGCTTCCGGTGACAGCACTGCTCCTCCCCTT | 27 |
| coding sequence | GGCGCTGTTGCTCCACGCAGCAAGGCCGGAAGTG | |
| | CAACTGGTCGAGAGCGGAGGCGGACTGGTGCAA | |
| | CCCGGCGGATCTCTGAGACTGAGCTGTGCCGCCT | |
| | CCGGCTTCACCTTCAACAAGAATGCCATGAACTG | |
| | GGTGAGACAAGCCCCCGGCAAGGGCCTCGAGTG | |
| | GGTGGGCAGAATTAGAAATAAGACCAACAACTA | |
| | TGCCACCTACTACGCTGACAGCGTGAAGGCTAGG | |
| | TTCACCATCTCTAGAGACGACTCCAAGAACTCTC | |
| | TGTATCTGCAGATGAACTCTCTGAAGACCGAGGA | |
| | CACCGCCGTGTATTACTGTGTGGCCGGCAATAGC | |
| | TTCGCCTATTGGGGACAAGGCACACTGGTGACCG | |
| | TGAGCGCTGGCGGAGGAGGAAGCGGAGGAGGAG | |
| | GCAGCGGAGGCGGCGGAAGCGATATCGTCATGA | |
| | CCCAGTCCCCCGACTCTCTGGCCGTCAGCCTCGG | |
| | CGAAAGAGCCACCATCAACTGCAAAAGCAGCCA | |
| | GTCTCTGCTGTACTCCAGCAACCAGAAGAACTAT | |
| | CTGGCTTGGTACCAGCAGAAACCCGGCCAGCCTC | |
| | CCAAACTGCTGATCTACTGGGCCAGCTCCAGAGA | |
| | ATCCGGCGTGCCCGATAGATTTTCCGGAAGCGGC | |
| | AGCGGCACCGATTTCACACTGACAATCAGCTCTC | |
| | TGCAAGCCGAGGATGTCGCTGTGTACTACTGCCA | |
| | GCAGTACTACAACTACCCTCTGACCTTCGGCCAA | |
| | GGCACCAAACTGGAAATCAAGAGTGCTGCTGCCT | |
| | TTGTCCCGGTATTTCTCCCAGCCAAACCGACCAC | |
| | GACTCCCGCCCCGCGCCCTCCGACACCCGCTCCC | |
| | ACCATCGCCTCTCAACCTCTTAGTCTTCGCCCCGA | |
| | GGCATGCCGACCCGCCGCCGGGGGTGCTGTTCAT | |
| | ACGAGGGGCTTGGACTTCGCTTGTGATATTTACA | |
| | TTTGGGCTCCGTTGGCGGGTACGTGCGGCGTCCTT | |
| | TTGTTGTCACTCGTTATTACTTTGTATTGTAATCA | |
| | CAGGAATCGCTCAAAGCGGAGTAGGTTGTTGCAT | |
| | TCCGATTACATGAATATGACTCCTCGCCGGCCTG | |
| | GGCCGACAAGAAAACATTACCAACCCTATGCCCC | |
| | CCCACGAGACTTCGCTGCGTACAGGTCCCGAGTG | |
| | AAGTTTTCCCGAAGCGCAGACGCTCCGGCATATC | |
| | AGCAAGGACAGAATCAGCTGTATAACGAACTGA | |
| | ATTTGGGACGCCGCGAGGAGTATGACGTGCTTGA | |
| | TAAACGCCGGGGGAGAGACCCGGAAATGGGGGG | |
| | TAAACCCCGAAGAAAGAATCCCCAAGAAGGACT | |
| | CTACAATGAACTCCAGAAGGATAAGATGGCGGA | |
| | GGCCTACTCAGAAATAGGTATGAAGGGCGAACG | |
| | ACGACGGGGAAAAGGTCACGATGGCCTCTACCA | |
| | AGGGTTGAGTACGGCAACCAAAGATACGTACGAT | |
| | GCACTGCATATGCAGGCCCTGCCTCCCAGA | |
| Anti-GPC3 CAR 1525b | MALPVTALLLPLALLLHAARPEVQLVESGGGLVQPGG | 28 |
| (VH/VL; | SLRLSCAASGFTFNKNAMNWVRQAPGKGLEWVGR | |
| 4-1BB CoS | IRNKTNNYATYYADSVKARFTISRDDSKNSLYLQM | |
| with signal peptide) | NSLKTEDTAVYYCVAGNSFAYWGQGTLVTVSAGG | |
| | GGSGGGGSGGGGSDIVMTQSPDSLAVSLGERATINC | |
| | KSSQSLLYSSNQKNYLAWYQQKPGQPPKLLIYWAS | |
| | SRESGVPDRFSGSGSGTDFTLTISSLQAEDVAVYYC | |
| | QQYYNYPLTFGQGTKLEIKSAAAFVPVFLPAKPTTT | |
| | PAPRPPTPAPTIASQPLSLRPEACRPAAGGAVHTRGL | |
| | DFACDIYIWAPLAGTCGVLLLSLVITLYCNHRNRKR | |
| | GRKKLLYIFKQPFMRPVQTTQEEDGCSCRFPEEEEG | |
| | GCELRVKFSRSADAPAYQQGQNQLYNELNLGRREE | |
| | YDVLDKRRGRDPEMGGKPRRKNPQEGLYNELQKD | |
| | KMAEAYSEIGMKGERRRGKGHDGLYQGLSTATKD | |
| | TYDALHMQALPPR | |
| Anti-GPC3 CAR 1525b | EVQLVESGGGLVQPGGSLRLSCAASGFTFNKNAMN | 29 |
| (no signal peptide) | WVRQAPGKGLEWVGRIRNKTNNYATYYADSVKA | |
| | RFTISRDDSKNSLYLQMNSLKTEDTAVYYCVAGNS | |
| | FAYWGQGTLVTVSAGGGGSGGGGSGGGGSDIVMT | |
| | QSPDSLAVSLGERATINCKSSQSLLYSSNQKNYLAW | |
| | YQQKPGQPPKLLIYWASSRESGVPDRFSGSGSGTDF | |
| | TLTISSLQAEDVAVYYCQQYYNYPLTFGQGTKLEIK | |
| | SAAAFVPVFLPAKPTTTPAPRPPTPAPTIASQPLSLRP | |
| | EACRPAAGGAVHTRGLDFACDIYIWAPLAGTCGVL | |
| | LLSLVITLYCNHRNRKRGRKKLLYIFKQPFMRPVQT | |
| | TQEEDGCSCRFPEEEEGGCELRVKFSRSADAPAYQQ | |
| | GQNQLYNELNLGRREEYDVLDKRRGRDPEMGGKP | |
| | RRKNPQEGLYNELQKDKMAEAYSEIGMKGERRRG | |
| | KGHDGLYQGLSTATKDTYDALHMQALPPR | |
| Anti-GPC3 CAR 1525b | ATGGCGCTTCCGGTGACAGCACTGCTCCTCCCCTT | 30 |
| coding sequence | GGCGCTGTTGCTCCACGCAGCAAGGCCGGAAGTG | |
| | CAACTGGTCGAGAGCGGAGGCGGACTGGTGCAA | |
| | CCCGGCGGATCTCTGAGACTGAGCTGTGCCGCCT | |
| | CCGGCTTCACCTTCAACAAGAATGCCATGAACTG | |
| | GGTGAGACAAGCCCCCGGCAAGGGCCTCGAGTG | |
| | GGTGGGCAGAATTAGAAATAAGACCAACAACTA | |
| | TGCCACCTACTACGCTGACAGCGTGAAGGCTAGG | |
| | TTCACCATCTCTAGAGACGACTCCAAGAACTCTC | |
| | TGTATCTGCAGATGAACTCTCTGAAGACCGAGGA | |
| | CACCGCCGTGTATTACTGTGTGGCCGGCAATAGC | |
| | TTCGCCTATTGGGGACAAGGCACACTGGTGACCG | |
| | TGAGCGCTGGCGGAGGAGGAAGCGGAGGAGGAG | |
| | GCAGCGGAGGCGGCGGAAGCGATATCGTCATGA | |
| | CCCAGTCCCCCGACTCTCTGGCCGTCAGCCTCGG | |
| | CGAAAGAGCCACCATCAACTGCAAAAGCAGCCA | |
| | GTCTCTGCTGTACTCCAGCAACCAGAAGAACTAT | |
| | CTGGCTTGGTACCAGCAGAAACCCGGCCAGCCTC | |
| | CCAAACTGCTGATCTACTGGGCCAGCTCCAGAGA | |
| | ATCCGGCGTGCCCGATAGATTTTCCGGAAGCGGC | |
| | AGCGGCACCGATTTCACACTGACAATCAGCTCTC | |
| | TGCAAGCCGAGGATGTCGCTGTGTACTACTGCCA | |
| | GCAGTACTACAACTACCCTCTGACCTTCGGCCAA | |
| | GGCACCAAACTGGAAATCAAGAGTGCTGCTGCCT | |
| | TTGTCCCGGTATTTCTCCCAGCCAAACCGACCAC | |
| | GACTCCCGCCCCGCGCCCTCCGACACCCGCTCCC | |
| | ACCATCGCCTCTCAACCTCTTAGTCTTCGCCCCGA | |
| | GGCATGCCGACCCGCCGCCGGGGGTGCTGTTCAT | |
| | ACGAGGGGCTTGGACTTCGCTTGTGATATTTACA | |
| | TTTGGGCTCCGTTGGCGGGTACGTGCGGCGTCCTT | |
| | TTGTTGTCACTCGTTATTACTTTGTATTGTAATCA | |
| | CAGGAATCGCAAACGGGGCAGAAAGAAACTCCT | |
| | GTATATATTCAAACAACCATTTATGAGACCAGTA | |
| | CAAACTACTCAAGAGGAAGATGGCTGTAGCTGCC | |
| | GATTTCCAGAAGAAGAAGAAGGAGGATGTGAAC | |
| | TGCGAGTGAAGTTTTCCCGAAGCGCAGACGCTCC | |
| | GGCATATCAGCAAGGACAGAATCAGCTGTATAAC | |
| | GAACTGAATTTGGGACGCCGCGAGGAGTATGACG | |
| | TGCTTGATAAACGCCGGGGGAGAGACCCGGAAA | |
| | TGGGGGGTAAACCCCGAAGAAAGAATCCCCAAG | |
| | AAGGACTCTACAATGAACTCCAGAAGGATAAGAT | |
| | GGCGGAGGCCTACTCAGAAATAGGTATGAAGGG | |
| | CGAACGACGACGGGGAAAAGGTCACGATGGCCT | |
| | CTACCAAGGGTTGAGTACGGCAACCAAAGATACG | |
| | TACGATGCACTGCATATGCAGGCCCTGCCTCCCA | |
| | GA | |
| Anti-GPC3 CAR 1526 | MALPVTALLLPLALLLHAARPDIVMTQSPDSLAVSLG | 31 |
| (VL/VH; | ERATINCKSSQSLLYSSNQKNYLAWYQQKPGQPPK | |
| CD28 CoS | LLIYWASSRESGVPDRFSGSGSGTDFTLTISSLQAED | |
| with signal peptide) | VAVYYCQQYYNYPLTFGQGTKLEIKGGGGSGGGG | |
| | SGGGGSEVQLVESGGGLVQPGGSLRLSCAASGFTF | |
| | NKNAMNWVRQAPGKGLEWVGRIRNKTNNYATYY | |
| | ADSVKARFTISRDDSKNSLYLQMNSLKTEDTAVYY | |
| | CVAGNSFAYWGQGTLVTVSASAAAFVPVFLPAKPT | |
| | TTPAPRPPTPAPTIASQPLSLRPEACRPAAGGAVHTR | |
| | GLDFACDIYIWAPLAGTCGVLLLSLVITLYCNHRNR | |
| | SKRSRLLHSDYMNMTPRRPGPTRKHYQPYAPPRDF | |
| | AAYRSRVKFSRSADAPAYQQGQNQLYNELNLGRR | |
| | EEYDVLDKRRGRDPEMGGKPRRKNPQEGLYNELQ | |
| | KDKMAEAYSEIGMKGERRRGKGHDGLYQGLSTAT | |
| | KDTYDALHMQALPPR | |
| Anti-GPC3 CAR 1526 | DIVMTQSPDSLAVSLGERATINCKSSQSLLYSSNQK | 32 |
| (no signal peptide) | NYLAWYQQKPGQPPKLLIYWASSRESGVPDRESGS | |
| | GSGTDFTLTISSLQAEDVAVYYCQQYYNYPLTFGQ | |
| | GTKLEIKGGGGSGGGGSGGGGSEVQLVESGGGLVQ | |
| | PGGSLRLSCAASGFTFNKNAMNWVRQAPGKGLEW | |
| | VGRIRNKTNNYATYYADSVKARFTISRDDSKNSLY | |
| | LQMNSLKTEDTAVYYCVAGNSFAYWGQGTLVTVS | |
| | ASAAAFVPVFLPAKPTTTPAPRPPTPAPTIASQPLSLR | |
| | PEACRPAAGGAVHTRGLDFACDIYIWAPLAGTCGV | |
| | LLLSLVITLYCNHRNRSKRSRLLHSDYMNMTPRRPG | |
| | PTRKHYQPYAPPRDFAAYRSRVKFSRSADAPAYQQ | |
| | GQNQLYNELNLGRREEYDVLDKRRGRDPEMGGKP | |
| | RRKNPQEGLYNELQKDKMAEAYSEIGMKGERRRG | |
| | KGHDGLYQGLSTATKDTYDALHMQALPPR | |
| Anti-GPC3 CAR 1526 | ATGGCGCTTCCGGTGACAGCACTGCTCCTCCCCTT | 33 |
| coding sequence | GGCGCTGTTGCTCCACGCAGCAAGGCCGGATATT | |
| | GTGATGACCCAAAGCCCCGACTCTCTGGCTGTGT | |
| | CCCTCGGAGAGAGGGCCACCATCAACTGTAAGTC | |
| | CTCCCAGTCTCTGCTCTACAGCTCCAACCAGAAG | |
| | AACTATCTGGCTTGGTACCAGCAGAAGCCCGGCC | |
| | AACCCCCCAAGCTGCTGATCTACTGGGCTTCCTCT | |
| | AGAGAATCCGGAGTGCCCGACAGATTCAGCGGA | |
| | AGCGGCTCCGGCACCGATTTTACCCTCACCATCA | |
| | GCTCTCTGCAAGCCGAGGATGTCGCCGTGTACTA | |
| | CTGCCAGCAGTACTATAACTACCCCCTCACCTTCG | |
| | GCCAAGGCACCAAGCTGGAAATTAAAGGCGGCG | |
| | GAGGCTCCGGAGGAGGCGGCTCCGGCGGAGGAG | |
| | GCTCCGAAGTGCAACTGGTGGAATCCGGAGGAG | |
| | GACTGGTGCAACCCGGAGGATCTCTGAGACTGAG | |
| | CTGCGCCGCTAGCGGCTTCACCTTCAACAAGAAC | |
| | GCTATGAACTGGGTGAGGCAAGCCCCCGGCAAG | |
| | GGACTGGAATGGGTGGGAAGAATTAGAAACAAG | |
| | ACCAACAACTACGCCACCTACTACGCCGACTCCG | |
| | TGAAGGCCAGATTCACCATCTCTAGAGACGACAG | |
| | CAAGAACTCCCTCTATCTGCAGATGAACTCTCTG | |
| | AAGACCGAAGACACAGCCGTGTATTACTGCGTCG | |
| | CCGGCAACTCCTTTGCCTATTGGGGACAAGGCAC | |
| | ACTGGTGACAGTGTCCGCCAGTGCTGCTGCCTTT | |
| | GTCCCGGTATTTCTCCCAGCCAAACCGACCACGA | |
| | CTCCCGCCCCGCGCCCTCCGACACCCGCTCCCAC | |
| | CATCGCCTCTCAACCTCTTAGTCTTCGCCCCGAGG | |
| | CATGCCGACCCGCCGCCGGGGGTGCTGTTCATAC | |
| | GAGGGGCTTGGACTTCGCTTGTGATATTTACATTT | |
| | GGGCTCCGTTGGCGGGTACGTGCGGCGTCCTTTT | |
| | GTTGTCACTCGTTATTACTTTGTATTGTAATCACA | |
| | GGAATCGCTCAAAGCGGAGTAGGTTGTTGCATTC | |
| | CGATTACATGAATATGACTCCTCGCCGGCCTGGG | |
| | CCGACAAGAAAACATTACCAACCCTATGCCCCCC | |
| | CACGAGACTTCGCTGCGTACAGGTCCCGAGTGAA | |
| | GTTTTCCCGAAGCGCAGACGCTCCGGCATATCAG | |
| | CAAGGACAGAATCAGCTGTATAACGAACTGAATT | |
| | TGGGACGCCGCGAGGAGTATGACGTGCTTGATAA | |
| | ACGCCGGGGGAGAGACCCGGAAATGGGGGGTAA | |
| | ACCCCGAAGAAAGAATCCCCAAGAAGGACTCTA | |
| | CAATGAACTCCAGAAGGATAAGATGGCGGAGGC | |
| | CTACTCAGAAATAGGTATGAAGGGCGAACGACG | |
| | ACGGGGAAAAGGTCACGATGGCCTCTACCAAGG | |
| | GTTGAGTACGGCAACCAAAGATACGTACGATGCA | |
| | CTGCATATGCAGGCCCTGCCTCCCAGA | |
| Anti-GPC3 CAR 1526b | MALPVTALLLPLALLLHAARPDIVMTQSPDSLAVSLG | 34 |
| (VL/VH; | ERATINCKSSQSLLYSSNQKNYLAWYQQKPGQPPK | |
| 4-1BB CoS | LLIYWASSRESGVPDRFSGSGSGTDFTLTISSLQAED | |
| with signal peptide) | VAVYYCQQYYNYPLTFGQGTKLEIKGGGGSGGGG | |
| | SGGGGSEVQLVESGGGLVQPGGSLRLSCAASGFTF | |
| | NKNAMNWVRQAPGKGLEWVGRIRNKTNNYATYY | |
| | ADSVKARFTISRDDSKNSLYLQMNSLKTEDTAVYY | |
| | CVAGNSFAYWGQGTLVTVSASAAAFVPVFLPAKPT | |
| | TTPAPRPPTPAPTIASQPLSLRPEACRPAAGGAVHTR | |
| | GLDFACDIYIWAPLAGTCGVLLLSLVITLYCNHRNR | |
| | KRGRKKLLYIFKQPFMRPVQTTQEEDGCSCRFPEEE | |
| | EGGCELRVKFSRSADAPAYQQGQNQLYNELNLGRR | |
| | EEYDVLDKRRGRDPEMGGKPRRKNPQEGLYNELQ | |
| | KDKMAEAYSEIGMKGERRRGKGHDGLYQGLSTAT | |
| | KDTYDALHMQALPPR | |
| Anti-GPC3 CAR 1526b | DIVMTQSPDSLAVSLGERATINCKSSQSLLYSSNQK | 35 |
| (no signal peptide) | NYLAWYQQKPGQPPKLLIYWASSRESGVPDRESGS | |
| | GSGTDFTLTISSLQAEDVAVYYCQQYYNYPLTFGQ | |
| | GTKLEIKGGGGSGGGGSGGGGSEVQLVESGGGLVQ | |
| | PGGSLRLSCAASGFTFNKNAMNWVRQAPGKGLEW | |
| | VGRIRNKTNNYATYYADSVKARFTISRDDSKNSLY | |
| | LQMNSLKTEDTAVYYCVAGNSFAYWGQGTLVTVS | |
| | ASAAAFVPVFLPAKPTTTPAPRPPTPAPTIASQPLSLR | |
| | PEACRPAAGGAVHTRGLDFACDIYIWAPLAGTCGV | |
| | LLLSLVITLYCNHRNRKRGRKKLLYIFKQPFMRPVQ | |
| | TTQEEDGCSCRFPEEEEGGCELRVKFSRSADAPAYQ | |
| | QGQNQLYNELNLGRREEYDVLDKRRGRDPEMGGK | |
| | PRRKNPQEGLYNELQKDKMAEAYSEIGMKGERRR | |
| | GKGHDGLYQGLSTATKDTYDALHMQALPPR | |
| Anti-GPC3 CAR 1526b | ATGGCGCTTCCGGTGACAGCACTGCTCCTCCCCTT | 36 |
| coding sequence | GGCGCTGTTGCTCCACGCAGCAAGGCCGGATATT | |
| | GTGATGACCCAAAGCCCCGACTCTCTGGCTGTGT | |
| | CCCTCGGAGAGAGGGCCACCATCAACTGTAAGTC | |
| | CTCCCAGTCTCTGCTCTACAGCTCCAACCAGAAG | |
| | AACTATCTGGCTTGGTACCAGCAGAAGCCCGGCC | |
| | AACCCCCCAAGCTGCTGATCTACTGGGCTTCCTCT | |
| | AGAGAATCCGGAGTGCCCGACAGATTCAGCGGA | |
| | AGCGGCTCCGGCACCGATTTTACCCTCACCATCA | |
| | GCTCTCTGCAAGCCGAGGATGTCGCCGTGTACTA | |
| | CTGCCAGCAGTACTATAACTACCCCCTCACCTTCG | |
| | GCCAAGGCACCAAGCTGGAAATTAAAGGCGGCG | |
| | GAGGCTCCGGAGGAGGCGGCTCCGGCGGAGGAG | |
| | GCTCCGAAGTGCAACTGGTGGAATCCGGAGGAG | |
| | GACTGGTGCAACCCGGAGGATCTCTGAGACTGAG | |
| | CTGCGCCGCTAGCGGCTTCACCTTCAACAAGAAC | |
| | GCTATGAACTGGGTGAGGCAAGCCCCCGGCAAG | |
| | GGACTGGAATGGGTGGGAAGAATTAGAAACAAG | |
| | ACCAACAACTACGCCACCTACTACGCCGACTCCG | |
| | TGAAGGCCAGATTCACCATCTCTAGAGACGACAG | |
| | CAAGAACTCCCTCTATCTGCAGATGAACTCTCTG | |
| | AAGACCGAAGACACAGCCGTGTATTACTGCGTCG | |
| | CCGGCAACTCCTTTGCCTATTGGGGACAAGGCAC | |
| | ACTGGTGACAGTGTCCGCCAGTGCTGCTGCCTTT | |
| | GTCCCGGTATTTCTCCCAGCCAAACCGACCACGA | |
| | CTCCCGCCCCGCGCCCTCCGACACCCGCTCCCAC | |
| | CATCGCCTCTCAACCTCTTAGTCTTCGCCCCGAGG | |
| | CATGCCGACCCGCCGCCGGGGGTGCTGTTCATAC | |
| | GAGGGGCTTGGACTTCGCTTGTGATATTTACATTT | |
| | GGGCTCCGTTGGCGGGTACGTGCGGCGTCCTTTT | |
| | GTTGTCACTCGTTATTACTTTGTATTGTAATCACA | |
| | GGAATCGCAAACGGGGCAGAAAGAAACTCCTGT | |
| | ATATATTCAAACAACCATTTATGAGACCAGTACA | |
| | AACTACTCAAGAGGAAGATGGCTGTAGCTGCCGA | |
| | TTTCCAGAAGAAGAAGAAGGAGGATGTGAACTG | |
| | CGAGTGAAGTTTTCCCGAAGCGCAGACGCTCCGG | |
| | CATATCAGCAAGGACAGAATCAGCTGTATAACGA | |
| | ACTGAATTTGGGACGCCGCGAGGAGTATGACGTG | |
| | CTTGATAAACGCCGGGGGAGAGACCCGGAAATG | |
| | GGGGGTAAACCCCGAAGAAAGAATCCCCAAGAA | |
| | GGACTCTACAATGAACTCCAGAAGGATAAGATGG | |
| | CGGAGGCCTACTCAGAAATAGGTATGAAGGGCG | |
| | AACGACGACGGGGAAAAGGTCACGATGGCCTCT | |
| | ACCAAGGGTTGAGTACGGCAACCAAAGATACGT | |
| | ACGATGCACTGCATATGCAGGCCCTGCCTCCCAG | |
| | A | |

TABLE 2
sgRNA Sequences and Target Sequences

| Name | Unmodified Sequence | Modified Sequence |
|---|---|---|
| TRAC sgRNA | AGAGCAACAGUGCUGU | A*G*A*GCAACAGUGCUGU |
| | GGCCguuuuagagcuagaaauag | GGCCguuuuagagcuagaaauagca |
| | caaguuaaaauaaggcuaguccguu | aguuaaaauaaggcuaguccguuauca |
| | aucaacuugaaaaaguggcaccgag | acuugaaaaaguggcaccgagucggug |
| | ucggugcUUUU | cU*U*U*U |
| | (SEQ ID NO: 37) | (SEQ ID NO: 38) |
| TRAC sgRNA spacer | AGAGCAACAGUGCUGU | A*G*A*GCAACAGUGCUGU |
| | GGCC | GGCC |
| | (SEQ ID NO: 39) | (SEQ ID NO: 40) |
| β2M sgRNA | GCUACUCUCUCUUUCU | G*C*U*ACUCUCUCUUUCU |
| | GGCCguuuuagagcuagaaauag | GGCCguuuuagagcuagaaauagca |
| | caaguuaaaauaaggcuaguccguu | aguuaaaauaaggcuaguccguuauca |
| | aucaacuugaaaaaguggcaccgag | acuugaaaaaguggcaccgagucggug |
| | ucggugcUUUU | cU*U*U*U |
| | (SEQ ID NO: 41) | (SEQ ID NO: 42) |
| β2M sgRNA spacer | GCUACUCUCUCUUUCU | G*C*U*ACUCUCUCUUUCU |
| | GGCC | GGCC |
| | (SEQ ID NO: 43) | (SEQ ID NO: 44) |
| TGFbRII sgRNA | CCCCUACCAUGACUUU | C*C*C*CUACCAUGACUUU |
| | AUUCguuuuagagcuagaaaua | AUUCguuuuagagcuagaaauagca |
| | gcaaguuaaaauaaggcuaguccgu | aguuaaaauaaggcuaguccguuauca |
| | uaucaacuugaaaaaguggcaccga | acuugaaaaaguggcaccgagucggug |
| | gucggugcUUUU | cU*U*U*U |
| | (SEQ ID NO: 45) | (SEQ ID NO: 46) |
| TGFbRII sgRNA spacer | CCCCUACCAUGACUUU | C*C*C*CUACCAUGACUUU |
| | AUUC | AUUC |
| | (SEQ ID NO: 47) | (SEQ ID NO: 48) |
| Reg1 sgRNA | ACGACGCGUGGGUGGC | A*C*G*ACGCGUGGGUGGC |
| | AAGCguuuuagagcuagaaaua | AAGCguuuuagagcuagaaauagca |
| | gcaaguuaaaauaaggcuaguccgu | aguuaaaauaaggcuaguccguuauca |
| | uaucaacuugaaaaaguggcaccga | acuugaaaaaguggcaccgagucggug |
| | gucggugcUUUU | cU*U*U*U |
| | (SEQ ID NO: 49) | (SEQ ID NO: 50) |
| Reg1 sgRNA spacer | ACGACGCGUGGGUGGC | A*C*G*ACGCGUGGGUGGC |
| | AAGC | AAGC |
| | (SEQ ID NO: 51) | (SEQ ID NO: 52) |
| CBLB-T3 sgRNA | AGCAAGCUGCCGCAGA | A*G*C*AAGCUGCCGCAGA |
| | UCGCguuuuagagcuagaaauag | UCGCguuuuagagcuagaaauagca |
| | caaguuaaaauaaggcuaguccguu | aguuaaaauaaggcuaguccguuauca |
| | aucaacuugaaaaaguggcaccgag | acuugaaaaaguggcaccgagucggug |
| | ucggugcUUUU | cU*U*U*U |
| | (SEQ ID NO: 53) | (SEQ ID NO: 54) |
| CBLB-T3 sgRNA spacer | AGCAAGCUGCCGCAGA | A*G*C*AAGCUGCCGCAGA |
| | UCGC | UCGC |
| | (SEQ ID NO: 55) | (SEQ ID NO: 56) |

Target Sequences

| | | Target Sequence-Without |
|---|---|---|
| Guide Name | Target Sequence (PAM) | PAM |
| TRAC | AGAGCAACAGTGCTGTG | AGAGCAACAGTGCTGTGG |
| | GCC (TGG) | CC |
| | (SEQ ID NO: 57) | (SEQ ID NO: 58) |
| B2M | GCTACTCTCTCTTTCTGG | GCTACTCTCTCTTTCTGGC |
| | CC (TGG) | C |
| | (SEQ ID NO: 59) | (SEQ ID NO: 60) |
| TGFbRII | CCCCTACCATGACTTTA | CCCCTACCATGACTTTATT |
| | TTC (TGG) | C |
| | (SEQ ID NO: 61) | (SEQ ID NO: 62) |
| Reg1 | ACGACGCGTGGGTGGCA | ACGACGCGTGGGTGGCAA |
| | AGC (GGG) | GC |
| | (SEQ ID NO: 63) | (SEQ ID NO: 64) |
| CBLB-T3 | AGCAAGCTGCCGCAGAT | AGCAAGCTGCCGCAGATC |
| | CGC (AGG) | GC |
| | (SEQ ID NO: 65) | (SEQ ID NO: 66) |

Exemplary sgRNA Formula
nnnnnnnnnnnnnnnnnnnnguuuuagagcuagaaauagcaaguuaaaauaaggcuaguccguuaucaacuugaaaaagu
ggcaccgagucggugcuuuu (SEQ ID NO: 67)
nnnnnnnnnnnnnnnnnnnnguuuuagagcuagaaauagcaaguaaaauaaggcuaguccguuaucaacuugaaaaagu
ggcaccgagucggugc (SEQ ID NO: 68)
n(17-30)guuuuagagcuagaaauagcaaguuaaaauaaggcuaguccguuaucaacuugaaaaaguggcaccgagucgg
ugcu (1-8) (SEQ ID NO: 69)
Exemplary Cas9 sequence (spCas9)
MDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDK
VMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNG
RDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVILADA
NLDKVLSAYNKHRDKPIREQAENIIHLFTLTNLGAPAAFKYFDTTIDRKRYTSTKEVLD
ATLIHQSITGLYETRIDLSQLGGD (SEQ ID NO: 70)
*: 2′-O-methyl phosphorothioate residue
“n” refers to the spacer sequence at the 5′ end

TABLE 3
Exemplary Donor Template Sequences

| | | SEQ ID |
|---|---|---|
| Name | Sequence | NO: |
| TRAC-LHA | GAGATGTAAGGAGCTGCTGTGACTTGCTCAAGGCCTTAT | 71 |
| (800 bp) | ATCGAGTAAACGGTAGTGCTGGGGCTTAGACGCAGGTG | |
| | TTCTGATTTATAGTTCAAAACCTCTATCAATGAGAGAGC | |
| | AATCTCCTGGTAATGTGATAGATTTCCCAACTTAATGCC | |
| | AACATACCATAAACCTCCCATTCTGCTAATGCCCAGCCT | |
| | AAGTTGGGGAGACCACTCCAGATTCCAAGATGTACAGTT | |
| | TGCTTTGCTGGGCCTTTTTCCCATGCCTGCCTTTACTCTG | |
| | CCAGAGTTATATTGCTGGGGTTTTGAAGAAGATCCTATT | |
| | AAATAAAAGAATAAGCAGTATTATTAAGTAGCCCTGCAT | |
| | TTCAGGTTTCCTTGAGTGGCAGGCCAGGCCTGGCCGTGA | |
| | ACGTTCACTGAAATCATGGCCTCTTGGCCAAGATTGATA | |
| | GCTTGTGCCTGTCCCTGAGTCCCAGTCCATCACGAGCAG | |
| | CTGGTTTCTAAGATGCTATTTCCCGTATAAAGCATGAGA | |
| | CCGTGACTTGCCAGCCCCACAGAGCCCCGCCCTTGTCCA | |
| | TCACTGGCATCTGGACTCCAGCCTGGGTTGGGGCAAAGA | |
| | GGGAAATGAGATCATGTCCTAACCCTGATCCTCTTGTCC | |
| | CACAGATATCCAGAACCCTGACCCTGCCGTGTACCAGCT | |
| | GAGAGACTCTAAATCCAGTGACAAGTCTGTCTGCCTATT | |
| | CACCGATTTTGATTCTCAAACAAATGTGTCACAAAGTAA | |
| | GGATTCTGATGTGTATATCACAGACAAAACTGTGCTAGA | |
| | CATGAGGTCTATGGACTTCA | |
| EF1α | GGCTCCGGTGCCCGTCAGTGGGCAGAGCGCACATCGCC | 72 |
| | CACAGTCCCCGAGAAGTTGGGGGGAGGGGTCGGCAATT | |
| | GAACCGGTGCCTAGAGAAGGTGGCGCGGGGTAAACTGG | |
| | GAAAGTGATGTCGTGTACTGGCTCCGCCTTTTTCCCGAG | |
| | GGTGGGGGAGAACCGTATATAAGTGCAGTAGTCGCCGT | |
| | GAACGTTCTTTTTCGCAACGGGTTTGCCGCCAGAACACA | |
| | GGTAAGTGCCGTGTGTGGTTCCCGCGGGCCTGGCCTCTT | |
| | TACGGGTTATGGCCCTTGCGTGCCTTGAATTACTTCCACT | |
| | GGCTGCAGTACGTGATTCTTGATCCCGAGCTTCGGGTTG | |
| | GAAGTGGGTGGGAGAGTTCGAGGCCTTGCGCTTAAGGA | |
| | GCCCCTTCGCCTCGTGCTTGAGTTGAGGCCTGGCCTGGG | |
| | CGCTGGGGCCGCCGCGTGCGAATCTGGTGGCACCTTCGC | |
| | GCCTGTCTCGCTGCTTTCGATAAGTCTCTAGCCATTTAAA | |
| | ATTTTTGATGACCTGCTGCGACGCTTTTTTTCTGGCAAGA | |
| | TAGTCTTGTAAATGCGGGCCAAGATCTGCACACTGGTAT | |
| | TTCGGTTTTTGGGGCCGCGGGCGGCGACGGGGCCCGTGC | |
| | GTCCCAGCGCACATGTTCGGCGAGGCGGGGCCTGCGAG | |
| | CGCGGCCACCGAGAATCGGACGGGGGTAGTCTCAAGCT | |
| | GGCCGGCCTGCTCTGGTGCCTGGCCTCGCGCCGCCGTGT | |
| | ATCGCCCCGCCCTGGGCGGCAAGGCTGGCCCGGTCGGC | |
| | ACCAGTTGCGTGAGCGGAAAGATGGCCGCTTCCCGGCCC | |
| | TGCTGCAGGGAGCTCAAAATGGAGGACGCGGCGCTCGG | |
| | GAGAGCGGGCGGGTGAGTCACCCACACAAAGGAAAAGG | |
| | GCCTTTCCGTCCTCAGCCGTCGCTTCATGTGACTCCACG | |
| | GAGTACCGGGCGCCGTCCAGGCACCTCGATTAGTTCTCG | |
| | AGCTTTTGGAGTACGTCGTCTTTAGGTTGGGGGGAGGGG | |
| | TTTTATGCGATGGAGTTTCCCCACACTGAGTGGGTGGAG | |
| | ACTGAAGTTAGGCCAGCTTGGCACTTGATGTAATTCTCC | |
| | TTGGAATTTGCCCTTTTTGAGTTTGGATCTTGGTTCATTC | |
| | TCAAGCCTCAGACAGTGGTTCAAAGTTTTTTTCTTCCATT | |
| | TCAGGTGTCGTGA | |
| Synthetic | AATAAAATCGCTATCCATCGAAGATGGATGTGTGTTGGT | 73 |
| poly(A) | TTTTTGTGTG | |
| signal | | |
| TRAC-RHA | TGGAGCAACAAATCTGACTTTGCATGTGCAAACGCCTTC | 74 |
| (800 bp) | AACAACAGCATTATTCCAGAAGACACCTTCTTCCCCAGC | |
| | CCAGGTAAGGGCAGCTTTGGTGCCTTCGCAGGCTGTTTC | |
| | CTTGCTTCAGGAATGGCCAGGTTCTGCCCAGAGCTCTGG | |
| | TCAATGATGTCTAAAACTCCTCTGATTGGTGGTCTCGGC | |
| | CTTATCCATTGCCACCAAAACCCTCTTTTTACTAAGAAA | |
| | CAGTGAGCCTTGTTCTGGCAGTCCAGAGAATGACACGGG | |
| | AAAAAAGCAGATGAAGAGAAGGTGGCAGGAGAGGGCA | |
| | CGTGGCCCAGCCTCAGTCTCTCCAACTGAGTTCCTGCCT | |
| | GCCTGCCTTTGCTCAGACTGTTTGCCCCTTACTGCTCTTC | |
| | TAGGCCTCATTCTAAGCCCCTTCTCCAAGTTGCCTCTCCT | |
| | TATTTCTCCCTGTCTGCCAAAAAATCTTTCCCAGCTCACT | |
| | AAGTCAGTCTCACGCAGTCACTCATTAACCCACCAATCA | |
| | CTGATTGTGCCGGCACATGAATGCACCAGGTGTTGAAGT | |
| | GGAGGAATTAAAAAGTCAGATGAGGGGTGTGCCCAGAG | |
| | GAAGCACCATTCTAGTTGGGGGAGCCCATCTGTCAGCTG | |
| | GGAAAAGTCCAAATAACTTCAGATTGGAATGTGTTTTAA | |
| | CTCAGGGTTGAGAAAACAGCTACCTTCAGGACAAAAGT | |
| | CAGGGAAGGGCTCTCTGAAGAAATGCTACTTGAAGATA | |
| | CCAGCCCTACCAAGGGCAGGGAGAGGACCCTATAGAGG | |
| | CCTGGGACAGGAGCTCAATGAGAAAGG | |

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B. D. Hames & S. J. Higgins eds. (1985»; Transcription and Translation (B. D. Hames & S. J. Higgins, eds. (1984; Animal Cell Culture (R. I. Freshney, ed. (1986»; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

Some aspects of the embodiments discussed above are disclosed in further detail in the following examples, which are not in any way intended to limit the scope of the present disclosure.

### Example 1. Production and Characterization of TRAC⁻/B2M⁻/Anti-Glypican 3 (GPC3)

CAR+ T Cells

This example describes generation and characterization of allogeneic human T cells that lack expression of the TRAC gene and β2M gene and express a chimeric antigen receptor (CAR) targeting Glypican 3 (GPC3) (anti-GPC3 CAR).

### Generation of Anti-GPC3 CAR-T Cells

Six unique exemplary GPC3 binders with either a CD28 costimulatory domain or a 4-1BB costimulatory domain were generated as presented in Table 4.

TABLE 4
Allogeneic CAR-T Cells with GPC3 constructs

| | | Costimulatory | SEQ |
|---|---|---|---|
| Cells | GPC3 binder | domain | ID ID |
| 1524 | HN3 single domain | CD28 | 10 |
| 1524b | HN3 single domain | 4-1BB | 13 |
| 1525 | hYP7vHvL | CD28 | 25 |
| 1525b | hYP7vHvL | 4-1BB | 28 |
| 1526 | hYP7vLvH | CD28 | 31 |
| 1526b | hYP7vLvH | 4-1BB | 34 |

Briefly, PBMCs were thawed and activated with TransAct™. After 0-3 days, the cells were electroporated with Cas9:sgRNA RNP complexes and transduced with adeno-associated adenoviral vectors (AAVs) to generate genetically engineered TRAC⁻/B2M⁻/anti-GPC3 CAR⁺ T cells, in which the nucleic acid encoding the anti-GPC3 CAR is inserted at the TRAC locus.

The sgRNAs, which form RNPs with the Cas9 enzyme, were introduced into the T cells in single or multiple electroporation events. After the electroporation, the cells were transduced with recombinant AAVs to introduce the donor template encoding the anti-GPC3 CAR. Recombinant AAV serotype 6 (AAV6) comprising one of the nucleotide sequences encoding an anti-GPC3 CAR listed in Table 1 above was delivered with Cas9:sgRNA RNPs (1 μM Cas9, 5 μM gRNA) to activated human T cells. The following sgRNAs were used: TRAC (SEQ ID NO: 38), and β2M (SEQ ID NO: 42). In general, unmodified or modified versions of the sgRNAs may be used. Exemplary gRNA sequences are shown in Table 2.

### Assessment of CAR Expression and Editing Efficiency

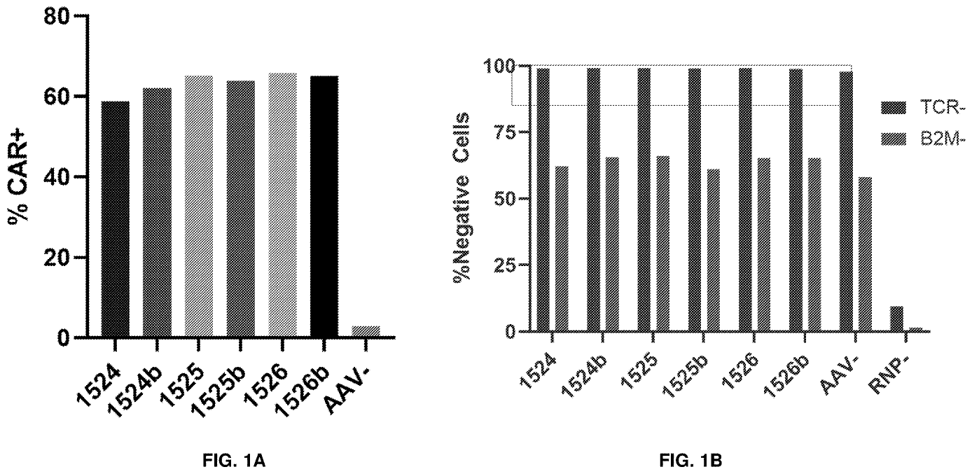

CAR expression was assessed by flow cytometry using biotinylated GPC3 antigen (1 pg), followed by incubation with APC-conjugated streptavidin (200 ng). As shown in FIG. 1A, CAR expression was similar across all anti-GPC3 CAR constructs. Editing efficiency for TRAC and β2M knockouts was assessed by flow cytometry and TIDE analysis. As shown in FIG. 1B, similar levels of editing efficiency for TRAC and β2M genes were observed in CAR-T cells expressing the anti-GPC3 CAR constructs noted above.

### Assessment of CD4:CD8 T Cell Ratios

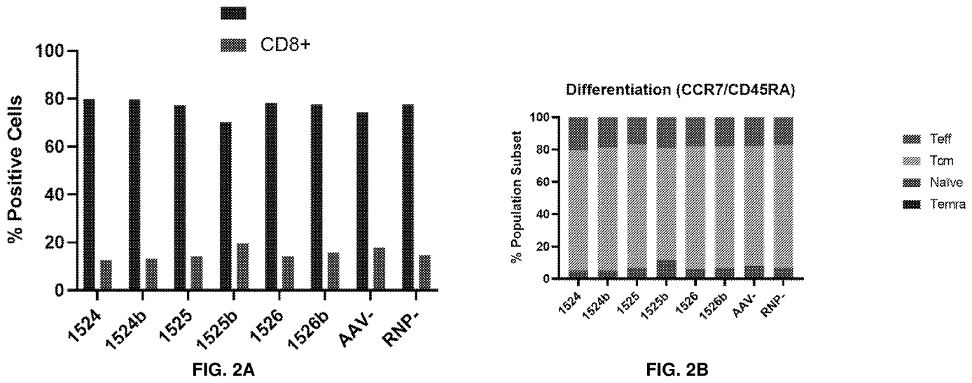

The frequency of CD4 and CD8 T cells in these cell cohorts, as well as differential profiles of the CAR T cells, were also determined by flow cytometry. Average frequencies are enumerated as shown in FIGS. 2A and 2B. There were no significant changes in CD4+ to CD8+ cell ratios in any of the CAR T cell populations. FIG. 2A. All cell populations were highly enriched for central memory cells. FIG. 2B.

### Assessment of Immune Checkpoint Molecules

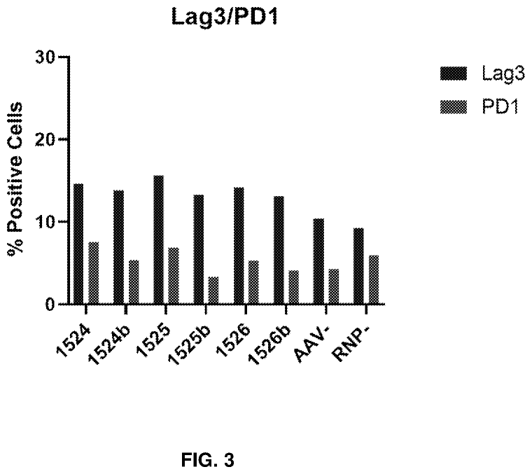

The expression of two immune checkpoint molecules, PD1 and Lag3, were evaluated in the cell populations. PD1 as an immune checkpoint molecule down-regulates T cell activity during immune responses to prevent autoimmune tissue damage (Jubel et al., Front. Immunol. 2020). Lymphocyte activation gene-3 (LAG-3) is an important immune checkpoint with relevance in cancer, infectious disease, and autoimmunity (Graydon et al., Front. Immunol. 2021). As a coinhibitory immune checkpoint, LAG3 inhibits the activation of its host cell and generally promotes a more suppressive immune response. No significant changes in PD1 and LAG-3 levels were observed in any of the cell populations as shown in FIG. 3 and Table 5

TABLE 5
PD1 and LAG3 Expression in CAR-T Cells
Expressing Anti-GPC3 CAR

| Cells | Lag-3+ (%) | PD1+ (%) |
|---|---|---|
| 1524 | 15 | 8 |
| 1524b | 14 | 5 |
| 1525 | 16 | 7 |
| 1525b | 13 | 3 |
| 1526 | 14 | 5 |
| 1526b | 13 | 4 |

### Example 2. In Vitro and In Vivo Cytotoxicity of Anti-GPC3 CAR T Cells

This example shows the ability of the anti-GPC3 CAR T cells described in Example 1 to selectively lyse GPC3+ cancer cells, in vitro and in vivo.

### In Vitro Cytotoxicity

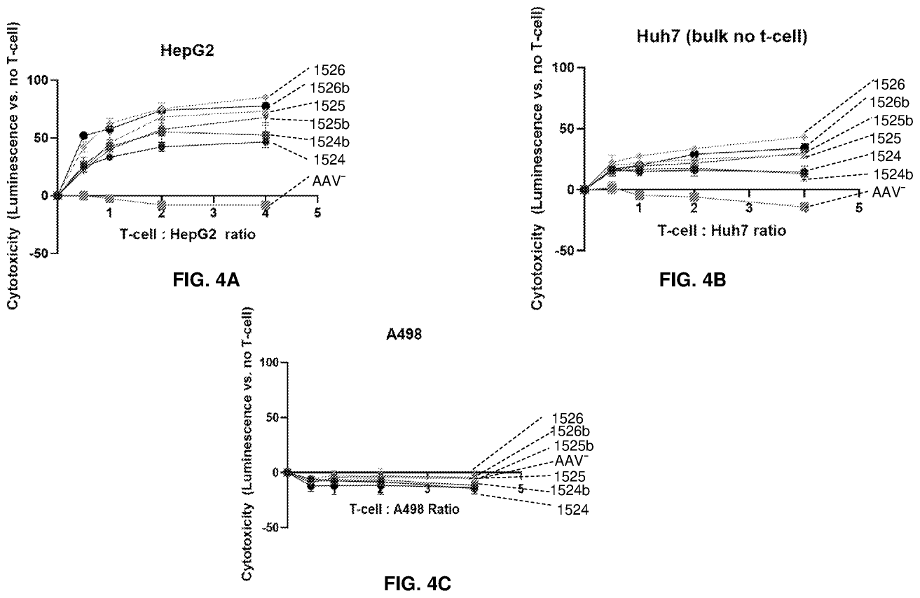

Anti-GPC3 CAR T cells were plated at different ratios with HepG2 target cells that have high GPC3 expression, with A498 cells that do not express GPC3, or with Huh7 cells that express low levels of GPC3. One day later, the number of viable target cells and T cells were counted. As shown in FIGS. 4A-4C, the CAR T cells specifically killed target cells that express the GPC3 antigen (HepG2 and Huh7 cells) but not GPC3 negative target cells (A498 cells). High cytotoxicity was observed when the target cells express a high GPC3 levels (e.g., the HepG2 target cells) as compared to target cells expressing a low level of GPC3 (e.g., Huh7 target cells).

### In Vivo Cytotoxicity

In vivo potency of anti-GPC3 CAR T cells in reducing tumor growth was examined using a mouse xenograft model.

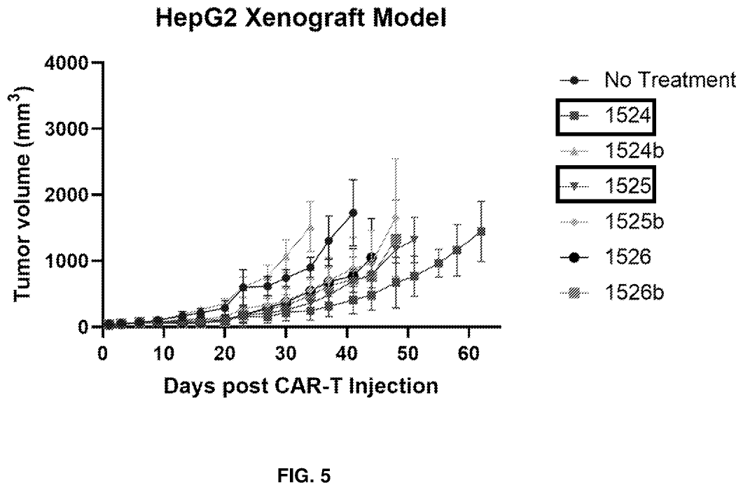

Female NSG mice were subcutaneously implanted on the right flank with HepG2 tumor cells (5×10⁶ cells) in 50% Matrigel®/50% media. When tumors were palpable, the mice were randomized into 5 groups and injected intravenously with CAR T cells (6×10⁶ CAR⁺ cells per mouse). Tumor volumes were evaluated every few days and are shown in FIG. 5 and Table 6. CAR-T cells expressing the 1524 and 1525 anti-GPC3 CAR showed the highest anti-tumor activity as compared with the other anti-GPC3 constructs. N/A indicates that there were no surviving mice for tumor volume measurements.

TABLE 6
Tumor Volume

| | Tumor Volumes (mm³) | | | | | | | | | | |
|---|---|---|---|---|---|---|---|---|---|---|---|
| | Day | Day | Day | Day | Day | Day | Day | Day | Day | Day | Day |
| Cohorts | 1 | 9 | 20 | 23 | 27 | 30 | 34 | 41 | 48 | 55 | 62 |
| No | 38 | 104 | 289 | 598 | 617 | 743 | 900 | 1728 | N/A | N/A | N/A |
| treat- | | | | | | | | | | | |
| ment | | | | | | | | | | | |
| 1524 | 38 | 77 | 109 | 161 | 152 | 226 | 245 | 408 | 673 | 965 | 1444 |
| 1524b | 38 | 102 | 356 | 603 | 760 | 1073 | 1517 | N/A | N/A | N/A | N/A |
| 1525 | 38 | 80 | 87 | 180 | 209 | 257 | 362 | 697 | 1178 | N/A | N/A |
| 1525b | 38 | 67 | 151 | 278 | 330 | 398 | 555 | 881 | 1656 | N/A | N/A |
| 1526 | 38 | 61 | 112 | 182 | 291 | 374 | 547 | 773 | N/A | N/A | N/A |
| 1526b | 38 | 75 | 87 | 181 | 273 | 324 | 480 | 720 | 1327 | N/A | N/A |

N/A = no surviving animals for measurements

### Example 3. Production and Characterization of CAR T Cell with Additional Potency

This example describes generation and characterization of allogeneic human TRAC⁻/B2M⁻/anti-GPC3 CAR⁺ T cells, which were additionally edited for the TGFBRII gene, Regnase-1 gene, and CBLB gene, as listed in Table 7.

TABLE 7
Genetically Engineered CAR-T Cells with GPC3 Constructs

| | | Co- | | Regnase- | |
|---|---|---|---|---|---|
| | GPC3 | stimulatory | TGFbR | 1 | CBLB |
| Cells | binder | domain | KO | KO | KO |
| 1524 | NH3 single | CD28 | No | No | No |
| | domain | | | | |
| 1524, TGFbR⁻, | NH3 single | CD28 | Yes | Yes | No |
| Regnase-1⁻ | domain | | | | |
| 1524, TGFbR⁻, | NH3 single | CD28 | Yes | No | Yes |
| CBLB⁻ | domain | | | | |
| 1525 | hYP7vHvL | CD28 | No | No | No |
| 1525, TGFbR⁻ | hYP7vHvL | CD28 | Yes | Yes | No |
| Regnase-1⁻ | | | | | |
| 1525, TGFbR⁻ | hYP7vHvL | CD28 | Yes | No | Yes |
| CBLB⁻ | | | | | |

Generation of TRAC⁻/B2M⁻/anti-GPC3 CAR T Cells with Additional Disruptions of TGFbRII/Reg-1 or TGFbRII/cbl-b

Briefly, the cells were electroporated with Cas9:sgRNA RNP complexes and transduced with adeno-associated adenoviral vectors (AAVs) to generate anti-GPC3 CAR T cells having TRAC, B2M, and TGFbRII knockouts, as well as CBLB, or Regnase-1 (REG-1) knockout. The sgRNAs, which form RNPs with the Cas9 enzyme were introduced into the T cells in single or multiple electroporation events. After electroporation, the cells were transduced with recombinant AAVs to introduce the donor template encoding the anti-GPC3 CAR (see Example 1 and Table 1). Recombinant AAV serotype 6 (AAV6) comprising one of the nucleotide sequences encoding one of the anti-GPC3 CARs was delivered with Cas9:sgRNA RNPs (1 μM Cas9, 5 μM gRNA) to activated human T cells. The following sgRNAs were used: TRAC (SEQ ID NO: 38), β2M (SEQ ID NO: 42), TGFbRII (SEQ ID NO: 46), REG-1 (SEQ ID NO: 50), and CBLB-T3 (SEQ ID NO: 54). Unmodified or modified versions of the sgRNAs may also be used. Exemplary gRNA sequences are shown in Table 2.

### Assessment of T-Cell Expansion

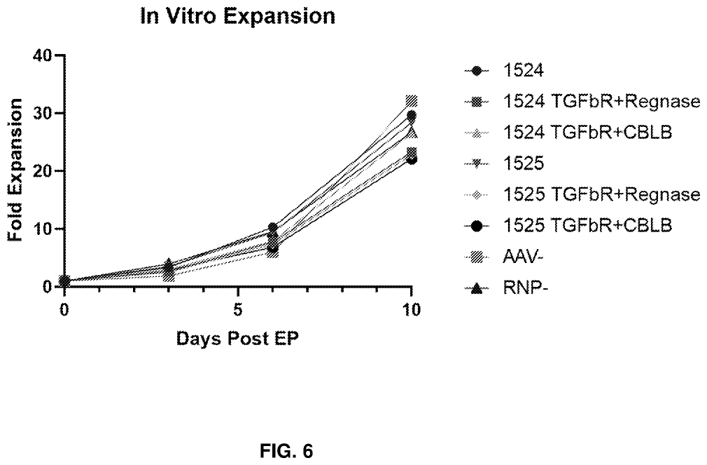

The engineered T cells were counted at regular intervals to assess T-cell expansion. As shown in FIG. 6 and Table 8, knocking out the TGFBRII gene, and the Regnase-1 gene or the CBLB gene had no impact on cell growth since the additionally edited cells expanded at similar rates as the TRAC⁻/B2M⁻/anti-GPC3 CAR⁺ cells.

TABLE 8
T-Cell Expansion

| | | 1524 | 1524 | | 1525 | 1525 | | |
|---|---|---|---|---|---|---|---|---|
| | | TGFbRII⁻ + | TGFbRII⁻ + | | TGFbRII⁻ + | TGFbRII⁻ + | | |
| Days | 1524 | Regnase-1⁻ | CBLB⁻ | 1525 | Regnase-1⁻ | CBLB⁻ | AAV⁻ | RNP⁻ |
| 3 | 3 | 3 | 3 | 4 | 3 | 3 | 2 | 4 |
| 6 | 10 | 8 | 8 | 9 | 7 | 7 | 6 | 10 |
| 10 | 30 | 23 | 27 | 28 | 23 | 22 | 32 | 27 |

### Assessment of CAR Expression and Editing Efficiency

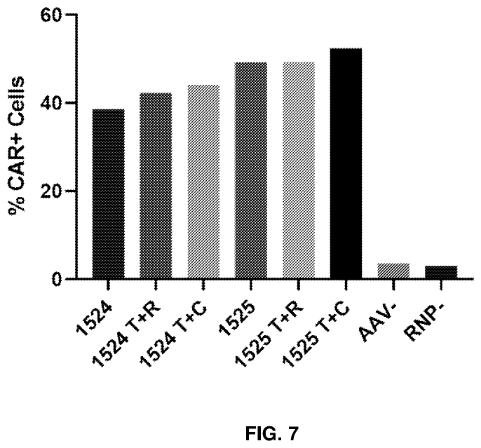

CAR expression was assessed by flow cytometry using biotinylated GPC3 antigen (1 μg), followed by incubation with APC-conjugated streptavidin (200 ng). CAR expression was similar across all anti-GPC3 CAR constructs. FIG. 7.

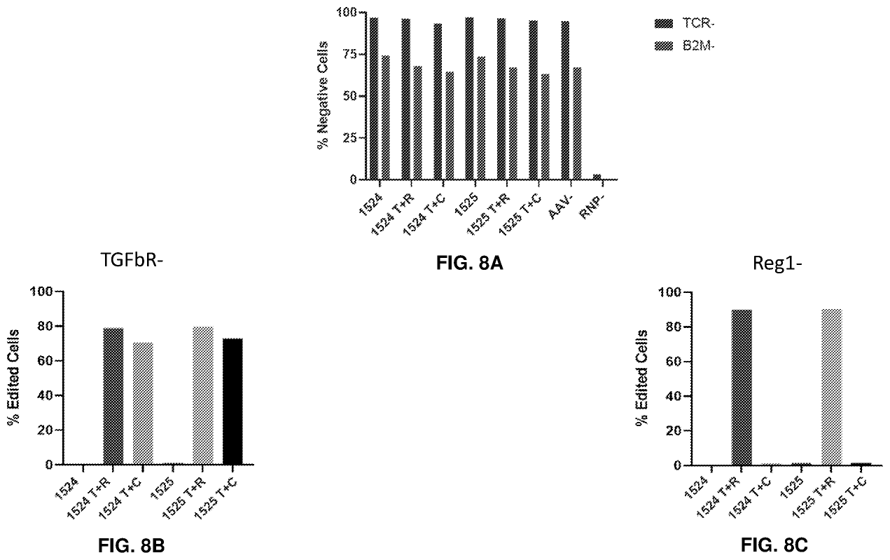

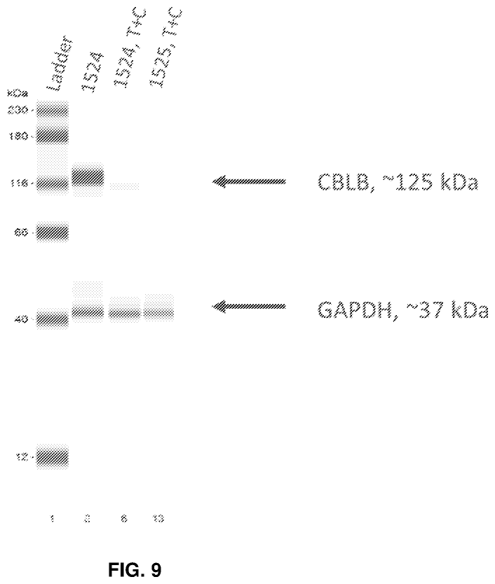

Editing efficiency for TRAC, β2M, TGFBRII, and Regnase-1 knockouts were assessed by flow cytometry and TIDE analysis. Similar levels of editing efficiencies were observed for TRAC and β2M in the presence or absence of Regnase-1 and TGFBRII. FIGS. 8A-8C Assessment of CBLB KO by western blot analysis showed that CBLB was efficiently knocked out in all anti-GPC3 CAR T cells examined. FIG. 9.

### Assessment of CD4:CD8 T Cell Ratios

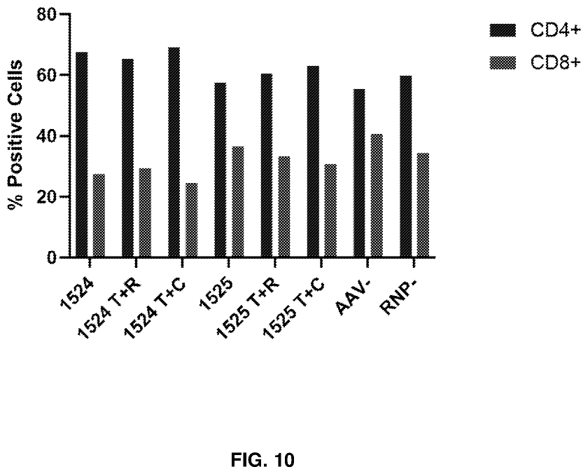

The frequency of CD4 and CD8 T cells in these cell cohorts was also determined by flow cytometry as shown in FIG. 10. Disruptions of Regnase-1 and TGFBRII genes showed no significant impact on CD4+ to CD8+ cell ratios in the CAR-T cell populations.

In sum, the results provided in this example indicate that genetically disrupting the TGFbRII gene, alone with disruption of either the Reg1 gene or the cbl-b gene, in anti-GPC3 CAR⁻T cells with disrupted TRAC, and B2M genes showed no significant impact on CAR-T cell properties.

### Example 4. In Vitro Cytotoxicity of Anti-GPC3 CAR T Cells with Potency Gene Edits

This example shows the ability of the anti-GPC3 CAR T cells described in Example 3 above to selectively lyse GPC3+ cancer cells.

### Study 1

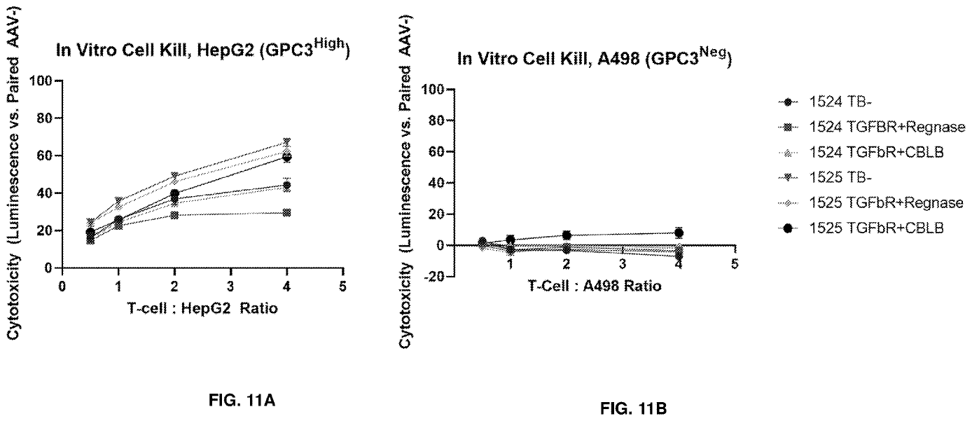

Anti-GPC3 CAR T cells were plated at different ratios with HepG2 target cells that have high GPC3 expression, or with A498 cells that do not express GPC3. One day later, the numbers of viable target cells and T cells was counted. The results are shown in FIGS. 11A-11B and Tables 9-10 below.

Cytotoxicity measurements were normalized as follows to account for loss of target cells during washing after co-culture:

Cytotoxicity_{CAR-Conc(A)}=100*(1−(Luminescence_{CAR-conc(A)}/Luminescence_{AAV-Conc(A)}),

Wherein cytotoxicity is a percentage measure of the inverse of the luminescence of ‘CAR-Conc(A)’, which refers to a given ratio of T cells to target cells within a CAR T cell population, divided by the luminescence of ‘AAV-Conc(A)’, which refers to a given ratio of T cells to target cells within the control (AAV-) population. Increased cell expansion was also observed when activated by GPC3+ target cells. No cytotoxicity against GPC3− target cells were observed as shown in Table 10 below.

TABLE 9
Cytotoxicity of HepG2 (GPC3^{high}) (%)

| Ratio | | 1524 | 1524 | | 1525 | 1525 |
|---|---|---|---|---|---|---|
| (T: | | TGFbRII⁻ | TGFbRII⁻, | | TGFbRII⁻ | TGFbRII⁻ |
| Target) | 1524 | Regnase-1⁻ | CBLB⁻ | 1525 | Regnase-1⁻ | CBLB⁻ |
| 0.5 | 16 | 15 | 16 | 24 | 24 | 19 |
| 1 | 26 | 23 | 24 | 36 | 33 | 26 |
| 2 | 37 | 28 | 35 | 49 | 46 | 40 |
| 4 | 44 | 30 | 43 | 67 | 62 | 59 |

TABLE 10
Cytotoxicity of A498 (GPC3⁻) (%)

| Ratio | | 1524 | 1524 | | 1525 | 1525 |
|---|---|---|---|---|---|---|
| (T: | | TGFbRII⁻ | TGFbRII⁻, | | TGFbRII⁻ | TGFbRII⁻ |
| Target) | 1524 | Regnase-1⁻ | CBLB⁻ | 1525 | Regnase-1⁻ | CBLB⁻ |
| 0.5 | 3 | 1 | 2 | 0 | 0 | 1 |
| 1 | 0 | 0 | 0 | 0 | 0 | 4 |
| 2 | 0 | 0 | 0 | 0 | 0 | 6 |
| 4 | 0 | 0 | 0 | 0 | 0 | 8 |

### Cytokine Secretion

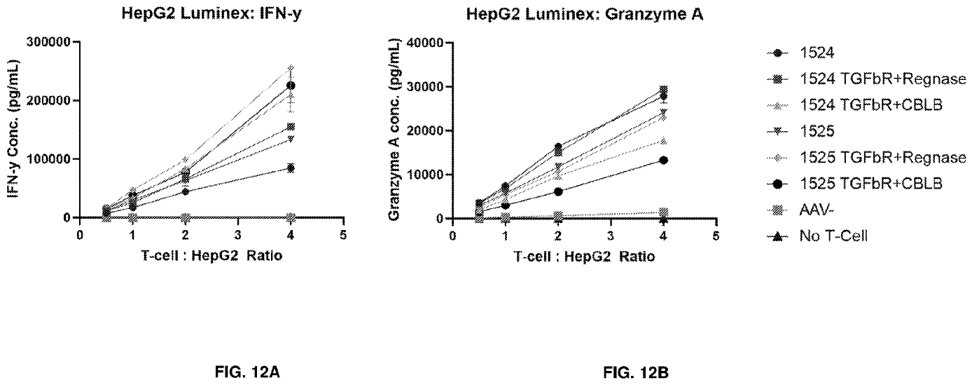

Cytokine secretion by these CAR T cells in the presence of GPC3 positive target cells was also measured. As shown in FIGS. 12A-12B and Tables 11-12 secretion of IFN-gamma and Granzyme A demonstrated a ratio-dependent increase.

TABLE 11
Secretion of IFN-gamma (pg/ml)

| | | 1524 | 1524 | | 1525 | 1525 | |
|---|---|---|---|---|---|---|---|
| Ratio | | TGFbRII⁻ | TGFbRII⁻ | | TGFbRII⁻ | TGFbRII⁻ | |
| (T:Target) | 1524 | Regnase-⁻ | CBLB⁻ | 1525 | Regnase-1⁻ | CBLB⁻ | AAV⁻ |
| 0.5 | 7659 | 11879 | 14647 | 11908 | 16166 | 14802 | 0 |
| 1 | 17065 | 25586 | 33725 | 30056 | 47208 | 37808 | 0 |
| 2 | 44473 | 66200 | 83528 | 63509 | 98994 | 77658 | 23 |
| 4 | 84975 | 155048 | 210456 | 133479 | 255890 | 225775 | 56 |

TABLE 12
Granzyme A (pg/ml)

| | | 1524 | 1524 | | 1525 | 1525 | |
|---|---|---|---|---|---|---|---|
| Ratio | | TGFbRII⁻ | TGFbRII⁻ | | TGFbRII⁻ | TGFbRII⁻ | |
| (T:Target) | 1524 | Regnase-1⁻ | CBLB⁻ | 1525 | Regnase-1⁻ | CBLB⁻ | AAV⁻ |
| 0.5 | 3596 | 3453 | 2021 | 3058 | 2571 | 1536 | 166 |
| 1 | 7480 | 6868 | 4363 | 5791 | 5530 | 3049 | 319 |
| 2 | 16445 | 15064 | 9729 | 11776 | 10730 | 6157 | 649 |
| 4 | 27857 | 29414 | 17832 | 24106 | 23010 | 13316 | 1418 |

### Study 2

The in vitro growth curves of cells with or without the potency edits were measured on various days post-electroporation. The data is presented in Table 13 and demonstrate that the cells were all in good condition.

TABLE 13
Fold-Expansion of Cells

| | | 1524 | 1524 | 1524 | | |
|---|---|---|---|---|---|---|
| Days | 1524 | TGFβR⁻ | Regnase⁻ | CBLB⁻ | AAV⁻ | RNP⁻ |
| 3 | 4 | 3 | 3 | 3 | 4 | 4 |
| 5 | 9 | 10 | 10 | 9 | 13 | 12 |
| 6 | 12 | 13 | 11 | 12 | 18 | 17 |
| 7 | 17 | 20 | 24 | 14 | 31 | 28 |
| 10 | 54 | 66 | 72 | 37 | 91 | 84 |

The cells with potency edits showed lower CAR knock-in and B2M knockout compared to the 1524 anti-GPC3 CAR T cells, and had similar TRAC-expressions. The ratio of CD4/CD8 cells in the cell populations were similar across all conditions.

The specific cytotoxicity of these cells to GPC3-expressing cells were measured using HepG2 target cells that have a high surface expression of GPC3. A498 cells that do not express GPC3 were used as a negative control. Cytotoxicity was measured using chemiluminescence vs. AAV⁻ cells and presented in Tables 14-15. All cells express good anti-GPC3-specific cytotoxicity. “E:T ratio” indicates effector cell to target cell ratio. “0” indicates that no cytotoxicity was observed.

TABLE 14
Cytotoxicity (% ) with HepG2 Cells (High GPC3-Expressing)

| E:T ratio | 1524 | 1524 TGFβR⁻ | 1524 Regnase⁻ | 1524 CBLB⁻ |
|---|---|---|---|---|
| 0.5 | 31 | 28 | 31 | 34 |
| 1 | 59 | 58 | 60 | 66 |
| 2 | 83 | 76 | 74 | 89 |
| 4 | 94 | 90 | 89 | 98 |

TABLE 15
Cytotoxicity (%) with A498 Cells (GPC3-Negative)

| E:T ratio | 1524 | 1524 TGFβR⁻ | 1524 Regnase⁻ | 1524 CBLB⁻ |
|---|---|---|---|---|
| 0.5 | 0 | 1 | 12 | 5 |
| 1 | 0 | 3 | 10 | 6 |
| 2 | 0 | 8 | 12 | 6 |
| 4 | 7 | 13 | 23 | 17 |

Hence, all the engineered cells showed good proliferation, specific cytotoxicity to the target, and were deemed phenotypically normal compared to equivalent CAR Tcells for use in an in vivo study.

### Example 5. In Vivo Cytotoxicity of Anti-GPC3 CAR T Cells with Potency Gene Edits

The engineered CAR T cells presented in the previous Examples were tested in a mouse model for hepatic carcinoma.

Briefly, 10×10⁶ Hep3B carcinoma cells were injected into the left flank of mice (5 per cohort) and allowed to grow up to ~125 mm³. Once the tumors reached the target volume, 8×10⁶ CAR T cells were administered intravenously. The mice were also rechallenged with another administration of tumor cells in the right flank 30 days after the administration of CAR T cells (day 54 after the initiation of the study). Any mouse that showed high tumor burdens were removed and euthanized. Groups with diminished mice numbers are denoted by an ‘*’. The tumor volumes of the primary tumors were measured at regular intervals and are presented in Table 16. The data indicates that the 1524 was efficacious against the primary tumor over the untreated control. Addition of potency edits, TGFBRII, Regnase-1, or CBLB, greatly improved primary tumor control of the 1524 CAR T cells.

TABLE 16
Primary Tumor Volumes (mm³)

| | No | | 1524 | 1524 | 1524 |
|---|---|---|---|---|---|
| Days | treatment | 1524 | TGFβR⁻ | Regnase⁻ | CBLB⁻ |
| 1 | 122 | 123 | 123 | 122 | 122 |
| 5 | 281 | 217 | 222 | 249 | 209 |
| 8 | 437 | 359 | 315 | 315 | 262 |
| 12 | 684 | 421 | 367 | 387 | 256 |
| 15 | 967 | 464 | 319 | 401 | 175 |
| 19 | 1535 | 598 | 231 | 379 | 143 |
| 22 | 2300 | 701 | 212 | 312 | 89 |
| 26 | 2784* | 875 | 191 | 292 | 64 |
| 29 | | 889 | 163 | 286 | 59 |
| 34 | | 815 | 165 | 277 | 53 |
| 37 | | 712 | 168 | 250 | 39 |
| 40 | | 639 | 145 | 200 | 17 |
| 43 | | 598 | 152 | 175 | 14 |
| 47 | | 540 | 152 | 168 | 15 |
| 50 | | 541 | 158 | 177 | 23 |
| 54 | | 593 | 171 | 181 | 24 |
| 57 | | 626 | 178 | 186 | 28 |
| 61 | | 750 | 198 | 255 | 35 |
| 64 | | 1058 | 225 | 341 | 36 |
| 68 | | 2133 | 383 | 556 | 30 |
| 71 | | 1210* | 367 | 600 | 27 |
| 75 | | 1806* | 380 | 609 | 24 |
| 78 | | 2156* | 382 | 762 | 27 |
| 82 | | | 913 | 1109 | 43 |
| 85 | | | 1370 | 1278 | 59 |
| 89 | | | 1442 | 1134 | 88 |
| 92 | | | 2071 | 1321* | 95 |
| 96 | | | | 1519* | 37 |
| 99 | | | | 1694* | 39 |
| 103 | | | | 1139* | 39 |
| 106 | | | | | 55 |
| 110 | | | | | 79 |
| 113 | | | | | 85 |
| 117 | | | | | 96 |
| 120 | | | | | 111 |
| 124 | | | | | 144 |
| 126 | | | | | 197 |

Tumor volumes of the second tumor administered as a rechallenge is presented in Table 17. In this case, a new mouse cohort was utilized as an untreated control since the untreated mice challenged with the primary tumor were all euthanized.

TABLE 17
Secondary (Rechallenge) Tumor Volumes (mm³)

| | No | | 1524 | 1524 | 1524 |
|---|---|---|---|---|---|
| Days | treatment | 1524 | TGFβR⁻ | Regnase⁻ | CBLB⁻ |
| 54 | 0 | 0 | 0 | 0 | 0 |
| 57 | 39 | 47 | 42 | 49 | 33 |
| 61 | 40 | 40 | 42 | 38 | 23 |
| 64 | 45 | 48 | 41 | 28 | 31 |
| 68 | 78 | 31 | 38 | 15 | 5 |
| 71 | 92 | 11* | 23 | 7 | 4 |
| 75 | 176 | 0* | 13 | 6 | 0 |
| 78 | 314 | 0)* | 13 | 5 | 0 |
| 82 | 728 | | 16 | 11 | 0 |
| 85 | 980 | | 0 | 10 | 0 |
| 89 | 1493 | | 0 | 10 | 0 |
| 92 | 1773 | | 0 | 13* | 7 |
| 96 | | | | 15* | 0 |
| 99 | | | | 9* | 0 |
| 103 | | | | 17* | 0 |
| 106 | | | | | 0 |
| 110 | | | | | 0 |
| 113 | | | | | 0 |
| 117 | | | | | 0 |
| 120 | | | | | 0 |
| 124 | | | | | 0 |
| 126 | | | | | 0 |

Body weights of the mice were monitored throughout the study. The data is presented in Table 18 as a measure of body weight changes from the start of the study.

TABLE 18
Mean Body Weight Change (%)

| | No | | 1524 | 1524 | 1524 |
|---|---|---|---|---|---|
| Days | Treatment | 1524 | TGFβR⁻ | Regnase⁻ | CBLB⁻ |
| 5 | 1.0 | −0.4 | 3.5 | 1.2 | 2.6 |
| 12 | −0.1 | 3.4 | 7.6 | 4.9 | 7.1 |
| 15 | −4.5 | 2.6 | 6.9 | 4.0 | 6.6 |
| 29 | | 4.8 | 10.0 | 5.9 | 7.1 |
| 40 | | 9.2 | 9.6 | 7.0 | 11.2 |
| 43 | | 8.8 | 7.9 | −1.5 | −0.5 |
| 47 | | 10.7 | 8.6 | 5.9 | 10.7 |
| 50 | | 12.2 | 5.4 | 6.8 | 10.5 |
| 54 | | 13.4 | 4.9 | 5.7 | 11.3 |
| 57 | | 10.7 | 8.7 | 2.7 | 11.9 |
| 61 | | 12.3 | 5.1 | 6.5 | 11.2 |
| 64 | | 7.9 | 0.7 | 3.8 | 9.9 |
| 68 | | 12.8 | −0.5 | 4.2 | 11.8 |
| 71 | | 13.5 | −6.2 | 1.6 | 8.5 |
| 75 | | 19.2 | −5.6 | 0.7 | 13.5 |
| 78 | | 17.4 | −10.1 | −3.1 | 10.2 |
| 82 | | | −3.7 | −4.5 | 10.1 |
| 85 | | | −7.2 | −4.7 | 8.0 |
| 89 | | | −6.7 | −8.3 | 4.6 |
| 92 | | | −5.7 | −7.5 | 4.0 |
| 96 | | | | −5.6 | 2.9 |
| 99 | | | | −4.8 | 6.3 |
| 103 | | | | −1.7 | 5.4 |
| 106 | | | | | 2.4 |
| 110 | | | | | 7.9 |
| 113 | | | | | 8.8 |
| 117 | | | | | 7.4 |
| 120 | | | | | 9.7 |
| 124 | | | | | 8.4 |
| 126 | | | | | 7.4 |

### Example 6. Generation and Characterization of Anti-GPC3 CAR-T Cells for Autologous Cell Therapy

This example describes construction and characterization of anti-GPC3 CAR-T cells for use in autologous cell therapy.

A lentiviral vector is constructed for delivering the coding sequence of an anti-GPC3 CAR (see Example 1 and Table 1) to immune cells for expression of the anti-GPC3 CAR. Briefly, a pCCL-c-MNDU3-X2 plasmid carrying a mutated WPRE (mWPRE) and the coding sequence of the anti-GPC3 CAR (as the gene of interest or GOI) is constructed. The general structure of the construct is shown below;
5′LTR-Gag′-RRE-cPPT-(promoter)-GOI-mWPRE-3-LTR

The plasmid carries a Moloney murine leukemia virus (MMLV) LTR (MNDU3) promoter that is known to be suitable for use in T cells and other leukocytes. The mWPRE can be obtained from a known plasmid carrying such (e.g., pENTR-L5-WPRE-L2, Addgene) or by mutagenesis of the wild type WPRE.

A desired cassette with “Anti-GPC3 CAR-mWPRE” or “MNDU3 promoter-Anti-GPC3 CAR-mWPRE” is first constructed on an intermediate plasmid. Briefly, following PCR amplification and DNA ligation of the cassette with the intermediate plasmid, the ligation mixture is used to transform E. coli cells. Clones generated are screened to select clones having plasmids with the desired cassette. This cassette is excised and cloned into the pCCL-c-MNDU3-X2 plasmid. After sequencing to verify insertion of the desired cassette, the resulting plasmid is used to transduce packaging cells, together with plasmids capable of producing proteins necessary for lentivirus packaging (e.g., gag/pol, Rev, and VSV-G) to produce lentivirus particles, which are then harvested.

Peripheral blood lymphocytes (PBLs) are obtained from a human subject needing elimination of GPC3⁺ cells. The cells are electroporated with Cas9:sgRNA RNP complexes for genetic editing of TGFbRII, Reg-1, and/or CBLB and transduced with the lentivirus described above, which carry a nucleic acid encoding the anti-GPC3 CAR construct. In some instances, the transduction is performed in the presence of RetroNectin to improve transduction efficiency. The sgRNA sequences for genetic editing of the TGFbRII, REG-1, and CBLB genes are shown in Table 2. The resultant anti-GPC3 CAR-T cells having one or more of disrupted TGFbRII, REG-1, and CBLB genes are collected.

The ability of these engineered anti-GPC3 CAR-T cells to kill GPC3+ target cells is examined using in vitro proliferation, cytotoxicity, and cytokine release assays; and in vivo cytotoxicity, persistence and cytokine analyses using NSG mouse models following the guidance provided in Examples 2-4 above. Cells recovered from mouse blood, spleens, and other tissues are used to determine engineered anti-GPC3 CAR cell persistence, biodistribution and phenotype using flow cytometry.

### Example 7. Autologous Cell Therapy Using Anti-GPC3 CAR-T Cells

This example describes autologous cell therapy of patients suffering from Hepatocellular Carcinoma (HCC) using anti-GPC3 CAR-T cells prepared from peripheral blood lymphocytes (PBIU) obtained from the patients.

PBLs are obtained from a patient having HCC and genetically engineered following the descriptions provided in Example 6 to produce engineered T cells expressing the anti-GPC3 CAR and having one or more of disrupted TGFbRII, REG-1, and CBLB genes.

The engineered anti-GCP3 CAR-T cells thus generated are administered to the patient. The ability of the anti-GPC3 CAR T cells to reduce tumor burden, persist in vivo, and differentiate in vivo are examined following conventional practices. Cells recovered from the blood are used to determine persistence, biodistribution and phenotype of the engineered anti-GPC3 CAR cells using flow cytometry.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A population of genetically engineered T cells, wherein the genetically engineered T cells comprise:
(a) a nucleic acid encoding a chimeric antigen receptor (CAR) that binds Glypican 3 (GPC3) (anti-GPC3 CAR); and
(b) one or more disrupted genes, which comprise:
(i) a disrupted T cell receptor alpha chain constant region (TRAC) gene,
(ii) a disrupted beta-2 microglobulin (β2M) gene,
(iii) a disrupted transforming growth factor beta receptor II (TGFbRII) gene,
(iv) a disrupted Regnase-1 (Reg1) gene,
(v) a disrupted Casitas B-Lineage Lymphoma Proto-Oncogene-B (CBLB) gene, or
(vi) a combination of any one of (i)-(v).

2. The population of genetically engineered T cells of claim 1, wherein the anti-GPC3 CAR comprises:
(a) an ectodomain that binds GPC3;
(b) a transmembrane domain; and
(c) an endodomain that comprises
(i) a co-stimulatory signaling domain and
(ii) a CD3ζ cytoplasmic signaling domain.

3. The population of genetically engineered T cells of claim 2, wherein the ectodomain comprises an anti-GPC3 fragment, which is an anti-GPC3 single domain antibody (VHH) or an anti-GPC3 single chain variable fragment (scFv).

4. The population of genetically engineered T cells of claim 3, wherein the ectodomain comprises the anti-GPC3 VHH, which comprises the same complementarity determining regions (CDRs) as those in SEQ ID NO: 9.

5. The population of genetically engineered T cells of claim 3, wherein the anti-GPC3 VHH comprises the amino acid sequence of SEQ ID NO: 9.

6. The population of genetically engineered T cells of claim 3, wherein the ectodomain comprises the anti-GPC3 scFv, which comprises a heavy chain variable (V_{H}) region comprising the same CDRs as those in SEQ ID NO: 22, and a light chain variable (V_{L}) region comprising the same CDRs as those in SEQ ID NO: 21.

7. The population of genetically engineered T cells of claim 6, wherein the anti-GPC3 scFv comprises the V_{H} set forth as SEQ ID NO: 22, and a V_{L} set forth as SEQ ID NO: 21.

8. The population of genetically engineered T cells of claim 7, wherein the anti-GPC3 scFv comprises the amino acid sequence of SEQ ID NO: 23, or SEQ ID NO: 24.

9. The population of genetically engineered T cells of claim 2, wherein the co-stimulatory domain is a CD28 co-stimulatory domain or a 4-1BB co-stimulatory domain.

10. The population of genetically engineered T cells of claim 2, wherein the transmembrane domain is a CD8 transmembrane domain.

11. The population of genetically engineered T cells of claim 2, wherein the anti-GPC3 CAR comprises an amino acid sequence of SEQ ID NOs: 11, 14, 26, 29, 32, or 35.

12. The population of genetically engineered T cells of claim 1, wherein the one or more disrupted genes of (b) comprises the disrupted TRAC gene, and the disrupted β2M gene.

13. The population of genetically engineered T cells of claim 12, wherein the one or more disrupted genes of (b) further comprises the disrupted TGFBRII gene, the disrupted Reg-1 gene, the disrupted CBLB gene, or a combination thereof.

14.-16. (canceled)

17. The population of genetically engineered T cells of claim 1, wherein the nucleic acid encoding the anti-GPC3 CAR is inserted in an endogenous genetic locus of the T cells.

18. The population of genetically engineered T cells of claim 17, wherein the endogenous genetic locus is within the disrupted TRAC gene, the disrupted β2M gene, the disrupted TGFbRII gene, the disrupted Reg-1 gene, or the disrupted CBLB gene.

19. The population of genetically engineered T cells of claim 18, wherein the endogenous genetic locus is within the disrupted TRAC gene.

20. The population of genetically engineered T cells of claim 19, wherein the disrupted TRAC gene comprises a deletion of SEQ ID NO: 58, which optionally is replaced by the nucleotide sequence encoding the anti-GPC3 CAR.

21. The population of genetically engineered T cells of claim 1, wherein the T cells are human T cells, which optionally are primary human T cells.

22.-41. (canceled)

42. A method of treating cancer in a subject, comprising administering to a subject in need thereof a population of genetically engineered T cells of claim 1.

43.-48. (canceled)

49. A chimeric antigen receptor that binds Glypican 3 (GPC3) (anti-GPC3 CAR), wherein the anti-GPC3 CAR is set forth in claim 2.

50.-54. (canceled)


SEQUENCE LISTING (75 sequences)

SEQ ID NO: 1 - protein, 21 aa, synthetic construct
MALPVTALLLPLALLLHAARP

SEQ ID NO: 2 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 3 - protein, 40 aa, synthetic construct
SKRSRLLHSDYMNMTPRRPGPTRKHYQPYAPPRDFAAYRS

SEQ ID NO: 4 - protein, 42 aa, synthetic construct
KRGRKKLLYIFKQPFMRPVQTTQEEDGCSCRFPEEEEGGCEL

SEQ ID NO: 5 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 4 aa, synthetic construct
SYEM

SEQ ID NO: 7 - protein, 16 aa, synthetic construct
SIYHSGSTYYNPSLKS

SEQ ID NO: 8 - protein, 8 aa, synthetic construct
VNMDRFDY

SEQ ID NO: 9 - protein, 116 aa, synthetic construct
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSS

SEQ ID NO: 10 - protein, 377 aa, synthetic construct
MALPVTALLLPLALLLHAARPQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 356 aa, synthetic construct
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1131 bp, synthetic construct
  source 1..1131: mol_type = other DNA
atggcgcttccggtgacagcactgctcctccccttggcgctgttgctccacgcagcaaggccgcaagtgcagctcgtgcaaagcggcggcggactggtgcaacccggcggatctctgagactgagctgcgccgccagctacttcgacttcgacagctacgagatgagctgggtgaggcaagcccccggaaaaggactggagtggatcggcagcatctaccacagcggctccacctactacaacccctctctgaaatctagagtgaccatctctagagacaactccaagaacacactgtacctccagatgaacacactgagagccgaggacaccgccacctattactgcgctagagtgaacatggacagattcgactactggggccaaggcacactggtgacagtgagctccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 13 - protein, 379 aa, synthetic construct
MALPVTALLLPLALLLHAARPQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 358 aa, synthetic construct
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 17 aa, synthetic construct
KSSQSLLYSSNQKNYLA

SEQ ID NO: 16 - protein, 7 aa, synthetic construct
WASSRES

SEQ ID NO: 17 - protein, 9 aa, synthetic construct
QQYYNYPLT

SEQ ID NO: 18 - protein, 5 aa, synthetic construct
KNAMN

SEQ ID NO: 19 - protein, 19 aa, synthetic construct
RIRNKTNNYATYYADSVKA

SEQ ID NO: 20 - protein, 6 aa, synthetic construct
GNSFAY

SEQ ID NO: 21 - protein, 113 aa, synthetic construct
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 22 - protein, 117 aa, synthetic construct
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 23 - protein, 245 aa, synthetic construct
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 24 - protein, 245 aa, synthetic construct
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKGGGGSGGGGSGGGGSEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 25 - protein, 506 aa, synthetic construct
MALPVTALLLPLALLLHAARPEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 485 aa, synthetic construct
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1518 bp, synthetic construct
  source 1..1518: mol_type = other DNA
atggcgcttccggtgacagcactgctcctccccttggcgctgttgctccacgcagcaaggccggaagtgcaactggtcgagagcggaggcggactggtgcaacccggcggatctctgagactgagctgtgccgcctccggcttcaccttcaacaagaatgccatgaactgggtgagacaagcccccggcaagggcctcgagtgggtgggcagaattagaaataagaccaacaactatgccacctactacgctgacagcgtgaaggctaggttcaccatctctagagacgactccaagaactctctgtatctgcagatgaactctctgaagaccgaggacaccgccgtgtattactgtgtggccggcaatagcttcgcctattggggacaaggcacactggtgaccgtgagcgctggcggaggaggaagcggaggaggaggcagcggaggcggcggaagcgatatcgtcatgacccagtcccccgactctctggccgtcagcctcggcgaaagagccaccatcaactgcaaaagcagccagtctctgctgtactccagcaaccagaagaactatctggcttggtaccagcagaaacccggccagcctcccaaactgctgatctactgggccagctccagagaatccggcgtgcccgatagattttccggaagcggcagcggcaccgatttcacactgacaatcagctctctgcaagccgaggatgtcgctgtgtactactgccagcagtactacaactaccctctgaccttcggccaaggcaccaaactggaaatcaagagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 28 - protein, 508 aa, synthetic construct
MALPVTALLLPLALLLHAARPEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 487 aa, synthetic construct
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 1524 bp, synthetic construct
  source 1..1524: mol_type = other DNA
atggcgcttccggtgacagcactgctcctccccttggcgctgttgctccacgcagcaaggccggaagtgcaactggtcgagagcggaggcggactggtgcaacccggcggatctctgagactgagctgtgccgcctccggcttcaccttcaacaagaatgccatgaactgggtgagacaagcccccggcaagggcctcgagtgggtgggcagaattagaaataagaccaacaactatgccacctactacgctgacagcgtgaaggctaggttcaccatctctagagacgactccaagaactctctgtatctgcagatgaactctctgaagaccgaggacaccgccgtgtattactgtgtggccggcaatagcttcgcctattggggacaaggcacactggtgaccgtgagcgctggcggaggaggaagcggaggaggaggcagcggaggcggcggaagcgatatcgtcatgacccagtcccccgactctctggccgtcagcctcggcgaaagagccaccatcaactgcaaaagcagccagtctctgctgtactccagcaaccagaagaactatctggcttggtaccagcagaaacccggccagcctcccaaactgctgatctactgggccagctccagagaatccggcgtgcccgatagattttccggaagcggcagcggcaccgatttcacactgacaatcagctctctgcaagccgaggatgtcgctgtgtactactgccagcagtactacaactaccctctgaccttcggccaaggcaccaaactggaaatcaagagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 31 - protein, 506 aa, synthetic construct
MALPVTALLLPLALLLHAARPDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKGGGGSGGGGSGGGGSEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSASAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 485 aa, synthetic construct
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKGGGGSGGGGSGGGGSEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSASAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1518 bp, synthetic construct
  source 1..1518: mol_type = other DNA
atggcgcttccggtgacagcactgctcctccccttggcgctgttgctccacgcagcaaggccggatattgtgatgacccaaagccccgactctctggctgtgtccctcggagagagggccaccatcaactgtaagtcctcccagtctctgctctacagctccaaccagaagaactatctggcttggtaccagcagaagcccggccaaccccccaagctgctgatctactgggcttcctctagagaatccggagtgcccgacagattcagcggaagcggctccggcaccgattttaccctcaccatcagctctctgcaagccgaggatgtcgccgtgtactactgccagcagtactataactaccccctcaccttcggccaaggcaccaagctggaaattaaaggcggcggaggctccggaggaggcggctccggcggaggaggctccgaagtgcaactggtggaatccggaggaggactggtgcaacccggaggatctctgagactgagctgcgccgctagcggcttcaccttcaacaagaacgctatgaactgggtgaggcaagcccccggcaagggactggaatgggtgggaagaattagaaacaagaccaacaactacgccacctactacgccgactccgtgaaggccagattcaccatctctagagacgacagcaagaactccctctatctgcagatgaactctctgaagaccgaagacacagccgtgtattactgcgtcgccggcaactcctttgcctattggggacaaggcacactggtgacagtgtccgccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 34 - protein, 508 aa, synthetic construct
MALPVTALLLPLALLLHAARPDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKGGGGSGGGGSGGGGSEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSASAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 487 aa, synthetic construct
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKGGGGSGGGGSGGGGSEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSASAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1524 bp, synthetic construct
  source 1..1524: mol_type = other DNA
atggcgcttccggtgacagcactgctcctccccttggcgctgttgctccacgcagcaaggccggatattgtgatgacccaaagccccgactctctggctgtgtccctcggagagagggccaccatcaactgtaagtcctcccagtctctgctctacagctccaaccagaagaactatctggcttggtaccagcagaagcccggccaaccccccaagctgctgatctactgggcttcctctagagaatccggagtgcccgacagattcagcggaagcggctccggcaccgattttaccctcaccatcagctctctgcaagccgaggatgtcgccgtgtactactgccagcagtactataactaccccctcaccttcggccaaggcaccaagctggaaattaaaggcggcggaggctccggaggaggcggctccggcggaggaggctccgaagtgcaactggtggaatccggaggaggactggtgcaacccggaggatctctgagactgagctgcgccgctagcggcttcaccttcaacaagaacgctatgaactgggtgaggcaagcccccggcaagggactggaatgggtgggaagaattagaaacaagaccaacaactacgccacctactacgccgactccgtgaaggccagattcaccatctctagagacgacagcaagaactccctctatctgcagatgaactctctgaagaccgaagacacagccgtgtattactgcgtcgccggcaactcctttgcctattggggacaaggcacactggtgacagtgtccgccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 37 - RNA, 100 nt, synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 38 - RNA, 100 nt, synthetic construct; identical to SEQ ID NO: 37
  source 1..100: mol_type = other RNA
  modified_base 1..4: mod_base = OTHER; note = modified with 2'-O-methyl phosphorothioate
  modified_base 97..100: mod_base = OTHER; note = modified with 2'-O-methyl phosphorothioate

SEQ ID NO: 39 - RNA, 20 nt, synthetic construct
  source 1..20: mol_type = other RNA
agagcaacagtgctgtggcc

SEQ ID NO: 40 - RNA, 20 nt, synthetic construct
  source 1..20: mol_type = other RNA
  modified_base 1..4: mod_base = OTHER; note = modified with 2'-O-methyl phosphorothioate
agagcaacagtgctgtggcc

SEQ ID NO: 41 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 42 - RNA, 100 nt, synthetic construct; identical to SEQ ID NO: 41
  source 1..100: mol_type = other RNA
  modified_base 1..4: mod_base = OTHER; note = modified with 2'-O-methyl phosphorothioate
  modified_base 97..100: mod_base = OTHER; note = modified with 2'-O-methyl phosphorothioate

SEQ ID NO: 43 - RNA, 20 nt, synthetic construct
  source 1..20: mol_type = other RNA
gctactctctctttctggcc

SEQ ID NO: 44 - RNA, 20 nt, synthetic construct
  source 1..20: mol_type = other RNA
  modified_base 1..4: mod_base = OTHER; note = modified with 2'-O-methyl phosphorothioate
gctactctctctttctggcc

SEQ ID NO: 45 - RNA, 100 nt, synthetic construct
  source 1..100: mol_type = other RNA
cccctaccatgactttattcgttttagagctagaaatagcaagttaaaataaggctagtccgttatcaacttgaaaaagtggcaccgagtcggtgctttt

SEQ ID NO: 46 - RNA, 100 nt, synthetic construct; identical to SEQ ID NO: 45
  source 1..100: mol_type = other RNA
  modified_base 1..4: mod_base = OTHER; note = modified with 2'-O-methyl phosphorothioate
  modified_base 97..100: mod_base = OTHER; note = modified with 2'-O-methyl phosphorothioate

SEQ ID NO: 47 - RNA, 20 nt, synthetic construct
  source 1..20: mol_type = other RNA
cccctaccatgactttattc

SEQ ID NO: 48 - RNA, 20 nt, synthetic construct
  source 1..20: mol_type = other RNA
  modified_base 1..4: mod_base = OTHER; note = modified with 2'-O-methyl phosphorothioate
cccctaccatgactttattc

SEQ ID NO: 49 - RNA, 100 nt, synthetic construct
  source 1..100: mol_type = other RNA
acgacgcgtgggtggcaagcgttttagagctagaaatagcaagttaaaataaggctagtccgttatcaacttgaaaaagtggcaccgagtcggtgctttt

SEQ ID NO: 50 - RNA, 100 nt, synthetic construct; identical to SEQ ID NO: 49
  source 1..100: mol_type = other RNA
  modified_base 1..4: mod_base = OTHER; note = modified with 2'-O-methyl phosphorothioate
  modified_base 97..100: mod_base = OTHER; note = modified with 2'-O-methyl phosphorothioate

SEQ ID NO: 51 - RNA, 20 nt, synthetic construct
  source 1..20: mol_type = other RNA
acgacgcgtgggtggcaagc

SEQ ID NO: 52 - RNA, 20 nt, synthetic construct
  source 1..20: mol_type = other RNA
  modified_base 1..4: mod_base = OTHER; note = modified with 2'-O-methyl phosphorothioate
acgacgcgtgggtggcaagc

SEQ ID NO: 53 - RNA, 100 nt, synthetic construct
  source 1..100: mol_type = other RNA
agcaagctgccgcagatcgcgttttagagctagaaatagcaagttaaaataaggctagtccgttatcaacttgaaaaagtggcaccgagtcggtgctttt

SEQ ID NO: 54 - RNA, 100 nt, synthetic construct; identical to SEQ ID NO: 53
  source 1..100: mol_type = other RNA
  modified_base 1..4: mod_base = OTHER; note = modified with 2'-O-methyl phosphorothioate
  modified_base 97..100: mod_base = OTHER; note = modified with 2'-O-methyl phosphorothioate

SEQ ID NO: 55 - RNA, 20 nt, synthetic construct
  source 1..20: mol_type = other RNA
agcaagctgccgcagatcgc

SEQ ID NO: 56 - RNA, 20 nt, synthetic construct
  source 1..20: mol_type = other RNA
  modified_base 1..4: mod_base = OTHER; note = modified with 2'-O-methyl phosphorothioate
agcaagctgccgcagatcgc

SEQ ID NO: 57 - DNA, 23 bp, synthetic construct
  source 1..23: mol_type = other DNA
agagcaacagtgctgtggcctgg

SEQ ID NO: 58 - DNA, 20 bp, synthetic construct
  source 1..20: mol_type = other DNA
agagcaacagtgctgtggcc

SEQ ID NO: 59 - DNA, 23 bp, synthetic construct
  source 1..23: mol_type = other DNA
gctactctctctttctggcctgg

SEQ ID NO: 60 - DNA, 20 bp, synthetic construct
  source 1..20: mol_type = other DNA
gctactctctctttctggcc

SEQ ID NO: 61 - DNA, 23 bp, synthetic construct
  source 1..23: mol_type = other DNA
cccctaccatgactttattctgg

SEQ ID NO: 62 - DNA, 20 bp, synthetic construct
  source 1..20: mol_type = other DNA
cccctaccatgactttattc

SEQ ID NO: 63 - DNA, 23 bp, synthetic construct
  source 1..23: mol_type = other DNA
acgacgcgtgggtggcaagcggg

SEQ ID NO: 64 - DNA, 20 bp, synthetic construct
  source 1..20: mol_type = other DNA
acgacgcgtgggtggcaagc

SEQ ID NO: 65 - DNA, 23 bp, synthetic construct
  source 1..23: mol_type = other DNA
agcaagctgccgcagatcgcagg

SEQ ID NO: 66 - DNA, 20 bp, synthetic construct
  source 1..20: mol_type = other DNA
agcaagctgccgcagatcgc

SEQ ID NO: 67 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 68 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 69 - RNA, 114 nt, synthetic construct
  source 1..114: mol_type = other RNA
  variation 17..30: note = n is a, c, g, u, or absent
  variation 108..114: note = u or absent
nnnnnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 70 - protein, 1368 aa, synthetic construct [sequence omitted]

SEQ ID NO: 71 - DNA, 800 bp, synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 72 - DNA, 1178 bp, synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 73 - DNA, 49 bp, synthetic construct
  source 1..49: mol_type = other DNA
aataaaatcgctatccatcgaagatggatgtgtgttggttttttgtgtg

SEQ ID NO: 74 - DNA, 804 bp, synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 75 - DNA, 1137 bp, synthetic construct
  source 1..1137: mol_type = other DNA
atggcgcttccggtgacagcactgctcctccccttggcgctgttgctccacgcagcaaggccgcaagtgcagctcgtgcaaagcggcggcggactggtgcaacccggcggatctctgagactgagctgcgccgccagctacttcgacttcgacagctacgagatgagctgggtgaggcaagcccccggaaaaggactggagtggatcggcagcatctaccacagcggctccacctactacaacccctctctgaaatctagagtgaccatctctagagacaactccaagaacacactgtacctccagatgaacacactgagagccgaggacaccgccacctattactgcgctagagtgaacatggacagattcgactactggggccaaggcacactggtgacagtgagctccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga